(19) United States
(12) Patent Application Publication
(10) Pub. No.: US 2026/0048052 A1
(43) Pub. Date: Feb. 19, 2026

(54) METHODS OF TREATING TENOSYNOVIAL GIANT CELL TUMORS

(71) Applicant: Deciphera Pharmaceuticals, LLC, Waltham, MA (US)

(72) Inventors: Rodrigo Ruiz Soto, Waltham, MA (US); Maitreyi G. Sharma, Waltham, MA (US)

(21) Appl. No.: 19/079,965

(22) Filed: Mar. 14, 2025

Related U.S. Application Data
(63) Continuation of application No. PCT/US2024/053261, filed on Oct. 28, 2024.
(60) Provisional application No. 63/594,018, filed on Oct. 29, 2023.
(60) Provisional application No. 63/622,689, filed on Jan. 19, 2024.
(60) Provisional application No. 63/574,671, filed on Apr. 4, 2024.
(60) Provisional application No. 63/650,146, filed on May 21, 2024.
(60) Provisional application No. 63/683,741, filed on Aug. 16, 2024.

(51) Int. Cl. A61K 31/513; A61P 35/00

(52) U.S. Cl. CPC A61K 31/513; A61P 35/00

(57) ABSTRACT

The present disclosure provides, inter alia, methods of treating a tenosynovial giant cell tumor in a patient in need thereof, where the methods comprise administering to the patient a compound represented by Formula (I):

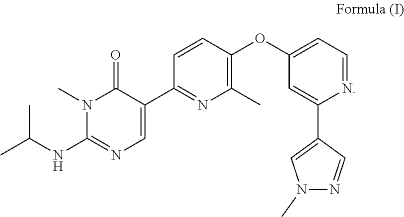 [SMILES: Cc1nc(-c2cnc(NC(C)C)n(C)c2=O)ccc1Oc1ccnc(-c2cnn(C)c2)c1]

### CROSS-REFERENCE

This application is a continuation of International Application No. PCT/US2024/053261 filed Oct. 28, 2024, which claims priority to U.S. Provisional Application No. 63/594,018 filed Oct. 29, 2023, U.S. Provisional Application No. 63/622,689 filed Jan. 19, 2024, U.S. Provisional Application No. 63/574,671 filed Apr. 4, 2024, U.S. Provisional Application No. 63/650,146 filed May 21, 2024, and U.S. Provisional Application No. 63/683,741 filed Aug. 16, 2024, the contents of each of which are incorporated herein by reference.

### BACKGROUND

Colony-stimulating factor 1 receptor (CSF1R) and its ligand, colony stimulating factor 1 (CSF1) together form a lineage dependency for normal macrophage development and differentiation from monocytes. As such, tumor-associated macrophages (TAMs) are dependent on CSF1R (also known as FMS) kinase activity for proliferation, and maintenance of their differentiated state and immunosuppressive phenotype. The role of TAMs in promoting an invasive and immunosuppressive tumor microenvironment is well established. TAMs mediate tumor growth, angiogenesis, invasiveness, metastasis, and immunosuppression through the secretion of and response to a variety of cytokines or other soluble factors. TAMs are educated by tumors to enable escape from immune surveillance by dampening a cytotoxic T cell immune response, thereby shielding the tumor from T cell eradication. For example, TAMs express PD-L1, a known immunosuppressive checkpoint that induces T cell anergy.

Several inhibitors targeting CSF1R have advanced into the clinic as direct antitumor therapies and potential immunotherapies. Many of these drugs also inhibit the closely related Type III tyrosine receptor kinases KIT, PDGFRα/β and FLT3, which may limit their utility due to off-target toxicity. Antibodies targeting CSF1R are much more specific yet result in >10,000-fold increases in plasma levels of CSF1, the ligand for CSF1R, due to blockade of CSF1 clearance, among other drawbacks.

Tenosynovial giant cell tumor (TGCT) is a proliferative and inflammatory disease that includes entities formerly known as pigmented villonodular synovitis (PVNS), and giant cell tumor of the tendon sheath (GCTTS), intraarticular or extraarticular. It is a rare neoplasm of the joint or tendon sheath, with destructive proliferation of synovial like mononuclear cells, admixed with multinucleate giant cells, foam cells, siderophages and inflammatory cells. There are two types of TGCT: the local or nodular form (where the tumor involves the tendons that support the joint, or in one area of the joint) and the diffuse form (where the entire lining of the joint is involved). Treatment is surgical excision of the tumor. However, it is often difficult to perform a marginal excision for the diffuse form of TGCT resulting in a high recurrence rate. It can be characterized by overexpression of CSF1.

There is a need for selective small-molecule CSF1R inhibitors that are useful in the treatment of disorders associated with the proliferation of TAMs including solid tumors of various cancers and treatment of mesenchymal tumors such as TGCT, including diffuse-type tenosynovial giant cell tumor (DTGCT) and localized tenosynovial giant cell tumor.

### SUMMARY

In one embodiment, described herein is a method of treating a tenosynovial giant cell tumor in a patient in need thereof and who is not amenable to surgical treatment of the tenosynovial giant cell tumor, comprising administering to the patient 30 mg, twice a week, of a compound represented by Formula (I):

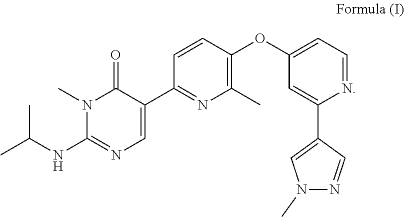 [SMILES: Cc1nc(-c2cnc(NC(C)C)n(C)c2=O)ccc1Oc1ccnc(-c2cnn(C)c2)c1]

In an additional embodiment, provided herein is a method of treating a tenosynovial giant cell tumor in a patient in need thereof, comprising administering to the patient 30 mg, twice a week, of a compound represented by Formula (I):

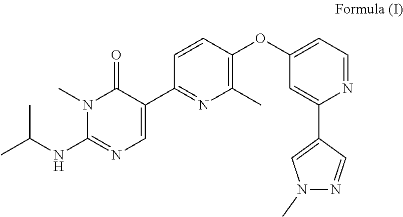 [SMILES: Cc1nc(-c2cnc(NC(C)C)n(C)c2=O)ccc1Oc1ccnc(-c2cnn(C)c2)c1]

wherein after at least 25 weeks of administering the compound, the patient achieves a partial response or a complete response as determined by RECIST v1.1.

In another embodiment, provided herein is a method of treating a tenosynovial giant cell tumor in a patient in need thereof, comprising administering to the patient 30 mg, twice a week, of a compound represented by Formula (I):

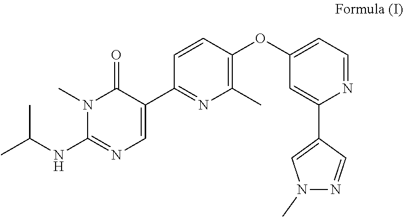 [SMILES: Cc1nc(-c2cnc(NC(C)C)n(C)c2=O)ccc1Oc1ccnc(-c2cnn(C)c2)c1]

wherein after at least 25 weeks of administering the compound, the patient achieves a partial response or a complete response as determined by Independent Radiologic Review (IRR) per tumor volume score (TVS).

In another embodiment, provided herein is a method of treating a tenosynovial giant cell tumor in a patient in need thereof, comprising administering to the patient 30 mg, twice a week, of a compound represented by:

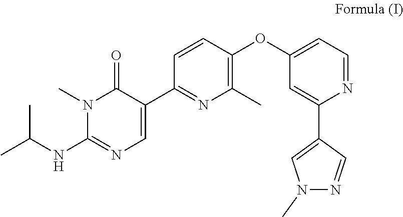 [SMILES: Cc1nc(-c2cnc(NC(C)C)n(C)c2=O)ccc1Oc1ccnc(-c2cnn(C)c2)c1]

wherein after at least 25 weeks of administering the compound, the patient has an improved active range of motion as compared to before administration of the compound to the patient.

In another embodiment, provided herein is a method of treating a tenosynovial giant cell tumor in a patient in need thereof, comprising administering to the patient 30 mg, twice a week, of a compound represented by Formula (I):

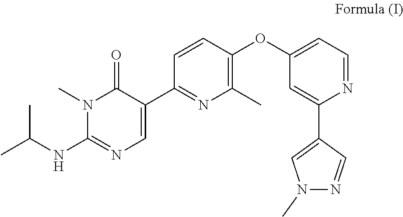 [SMILES: Cc1nc(-c2cnc(NC(C)C)n(C)c2=O)ccc1Oc1ccnc(-c2cnn(C)c2)c1]

wherein after at least 25 weeks of administering the compound, the patient achieves an improved PROMIS-PF score as compared to before administration of the compound to the patient.

In another embodiment, provided herein is a method of treating a tenosynovial giant cell tumor in a patient in need thereof, comprising administering to the patient 30 mg, twice a week, of a compound represented by Formula (I):

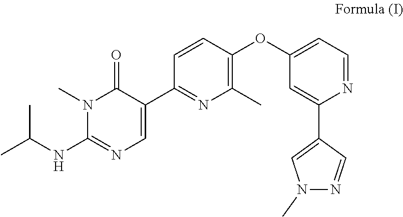 [SMILES: Cc1nc(-c2cnc(NC(C)C)n(C)c2=O)ccc1Oc1ccnc(-c2cnn(C)c2)c1]

wherein after at least 25 weeks of administering the compound, the patient achieves an improved score under the Worst Stiffness Numeric Rating Scale as compared to before administration of the compound to the patient.

In another embodiment, provided herein is a method of treating a tenosynovial giant cell tumor in a patient in need thereof, comprising administering to the patient 30 mg, twice a week, of a compound represented by Formula (I):

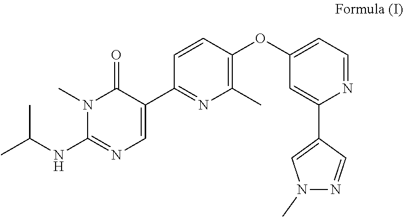 [SMILES: Cc1nc(-c2cnc(NC(C)C)n(C)c2=O)ccc1Oc1ccnc(-c2cnn(C)c2)c1]

wherein after at least 25 weeks of administering the compound, the patient achieves an improved score under the EQ-Visual Analog Scale (EQ-VAS) as compared to before administration of the compound to the patient.

In another embodiment, provided herein is a method of treating a tenosynovial giant cell tumor in a patient in need thereof, comprising administering to the patient 30 mg, twice a week, of a compound represented by Formula (I):

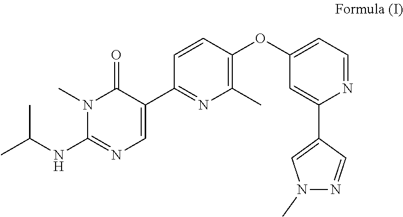 [SMILES: Cc1nc(-c2cnc(NC(C)C)n(C)c2=O)ccc1Oc1ccnc(-c2cnn(C)c2)c1]

wherein after at least 25 weeks of administering the compound, the patient achieves a greater than or equal to 30% reduction in score under the Brief Pain Inventory Numerical Rating Scale as compared to before administration of the compound to the patient.

In another embodiment, provided herein is a method of treating a patient having elevated levels of creatine phosphokinase (CPK) while being administered, for the treatment of a tenosynovial giant cell tumor, 30 mg, twice a week, of a compound represented by Formula (I):

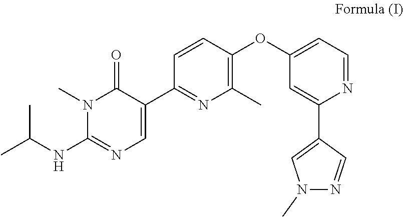 [SMILES: Cc1nc(-c2cnc(NC(C)C)n(C)c2=O)ccc1Oc1ccnc(-c2cnn(C)c2)c1]

the method comprising administering the compound to the patient in a dose that is less than 30 mg twice a week.

### BRIEF DESCRIPTION OF THE DRAWINGS

FIG. 1 shows duration of response by independent radiologic review (IRR) per RECIST v1.1 for responders in the study of Example 1 at 25 weeks for receiving the compound of Formula (I) (vimseltinib) at 30 mg twice a week.

FIG. 2 shows duration of response by independent radiologic review (IRR) per tumor volume score (TVS) for responders in the study of Example 1 at 25 weeks for receiving the compound of Formula (I) (vimseltinib) at 30 mg twice a week.

FIG. 3 shows the change from base in active range of motion (ROM) in patients receiving 30 mg twice a week of the compound of Formula (I) (vimseltinib), as compared to patients receiving placebo, in the study of Example 1 at 25 weeks.

FIG. 4 shows the change from base in PROMIS physical function (PROMIS-PF) in patients receiving 30 mg twice a week of the compound of Formula (I) (vimseltinib), as compared to patients receiving placebo, in the study of Example 1 at 25 weeks.

FIG. 5 shows the change from base in Worst Stiffness Numeric Rating Scale (NRS) in patients receiving 30 mg twice a week of the compound of Formula (I) (vimseltinib), as compared to patients receiving placebo, in the study of Example 1 at 25 weeks.

FIG. 6 shows the change from baseline in EQ-visual analogue scale (EQ-VAS) in patients receiving 30 mg twice a week of the compound of Formula (I) (vimseltinib), as compared to patients receiving placebo, in the study of Example 1 at 25 weeks.

FIG. 7A depicts best percent change in target lesions as assessed by IRR using RECIST v1.1 across patients in the study of Example 1. FIG. 7B depicts a Forest plot of objective response by patient subgroup.

FIG. 8 depicts the proportion of patients with a functional or symptomatic improvements in response to treatment with the compound of Formula (I) (vimseltinib) in the study of Example 1. The figure depicts the number of measures for which patients had a response to treatment, as described herein.

FIG. 9A and FIG. 9B depict changes from baseline in active range motion (ROM) for cohorts at Week 25 in the study of Example 2. Associations that are greater 100% are due to the reference ranges. Graphs show individual patient values. In cohort A (FIG. 9A), increase from baseline: n=23; no change: n=5; decrease: n=4. In cohort B (FIG. 9B), increase from baseline: n=9; no change: n=3; decrease: n=1.

FIG. 10 shows the percentage of treatment-emergent adverse events (TEAEs) by Maximum Grade in greater than or equal to 15% of patients occurring before and after 12 months on treatment of vimseltinib in the study of Example 2. The safety population includes patients who received at least 1 dose of study drug. Severity was assessed by the investigator according to the toxicity grade described in the National Cancer Institute CTCAE v4.03 (grade 1 [mild] to grade 5 [death]). Time denotes the earliest start date of the worst grade for each TEAE; if the highest grade TEAE occurred in the first 12 months of treatment, lower grade events that occurred after 12 months were not captured in this figure. Key: AST, aspartate aminotransferase; COVID-19, coronavirus disease 2019; CPK, creatine phosphokinase; CTCAE v4.03, Common Terminology Criteria for Adverse Events version 4.03; LDH, lactate dehydrogenase; TEAE, treatment-emergent adverse event.

### DETAILED DESCRIPTION

The features and other details of the disclosure will now be more particularly described. Certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Definitions

The compound represented by Formula (I) as described herein is also referred to as “vimseltinib.” The compound represented by Formula (I) as described herein also refers to 2-(isopropylamino)-3-methyl-5-(6-methyl-5-((2-(1-methyl-IH-pyrazol-4-yl)pyridin-4-yl)oxy)pyridin-2-yl)pyrimidin-4(3H)-one.

As used herein, the crystalline dihydrate form of the compound represented by Formula (I) is

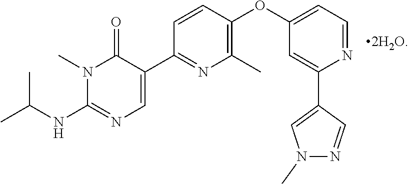 [SMILES: Cc1nc(-c2cnc(NC(C)C)n(C)c2=O)ccc1Oc1ccnc(-c2cnn(C)c2)c1]

The crystalline dihydrate form of the compound represented by Formula (I) is also referred to herein as “vimseltinib dihydrate.”

All ranges recited herein include the endpoints, including those that recite a range “between” two values. The terms “substantially” and “about” are each to be construed as modifying a term or value such that it is not an absolute. This includes, at very least, the degree of expected experimental error, technique error and instrument error for a given technique used to measure a value.

As used herein, the terms “individual,” “patient,” or “subject” are used interchangeably herein and include any animal, including mammals, including mice, rats, other rodents, rabbits, dogs, cats, swine, cattle, sheep, horses, or primates, and humans. The compounds described herein can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like). The mammal treated in the methods described herein is desirably a mammal in which treatment of a disorder described herein is desired, such as a human.

As used herein, “TGCT” refers to tenosynovial giant cell tumor.

As used herein, “DTGCT” refers to diffuse tenosynovial giant cell tumor.

As used herein, “treating” includes any effect, e.g., lessening, reducing, modulating, or eliminating, that results in the improvement of the condition, disease, disorder and the like.

“PROMIS-PF” as used herein means Patient Reported Outcomes Measurement Information System Physical Function.

A compound described herein, e.g., the compound of Formula (I), can be formulated as a pharmaceutical composition using a pharmaceutically acceptable carrier and administered by a variety of routes. In some embodiments, such compositions are for oral administration. In some embodiments, such compositions are for parenteral (by injection) administration (e.g., a composition formulated for local injection at the site of a tumor, e.g., a tenosynovial giant cell tumor, e.g., a diffuse tenosynovial giant cell tumor or a localized tenosynovial giant cell tumor). In some embodiments, such compositions are for transdermal administration. In some embodiments, such compositions are for intravenous (IV) administration. In some embodiments, such compositions are for intramuscular (IM) administration. Such pharmaceutical compositions and processes for preparing them are well known in the art. See, e.g., REMINGTON: THE SCIENCE AND PRACTICE OF PHARMACY (A. Gennaro, et al., eds., 19^{th} ed., Mack Publishing Co., 1995).

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications and this disclosure.

### Methods of Treatment

The present disclosure provides, in part, methods of treating tenosynovial giant cell tumors.

In one embodiment, described herein is a method of treating a tenosynovial giant cell tumor in a patient in need thereof and who is not amenable to surgical treatment of the tenosynovial giant cell tumor, comprising administering to the patient 30 mg, twice a week, of a compound represented by Formula (I):

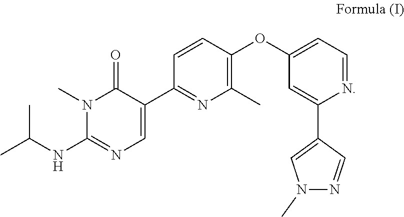 [SMILES: Cc1nc(-c2cnc(NC(C)C)n(C)c2=O)ccc1Oc1ccnc(-c2cnn(C)c2)c1]

In some embodiments, the patient does not qualify for surgery. In some embodiments, the patient is surgically ineligible (e.g., for surgical treatment of TGCT). In some embodiments, the surgical treatment is a high-risk procedure. In some embodiments, the patient has refused the surgical treatment.

In an additional embodiment, provided herein is a method of treating a tenosynovial giant cell tumor in a patient in need thereof, comprising administering to the patient 30 mg, twice a week, of a compound represented by Formula (I):

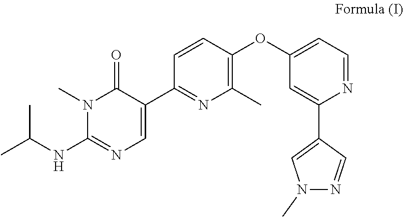 [SMILES: Cc1nc(-c2cnc(NC(C)C)n(C)c2=O)ccc1Oc1ccnc(-c2cnn(C)c2)c1]

wherein after at least 25 weeks of administering the compound, the patient achieves a partial response or a complete response as determined by RECIST v1.1.

In another embodiment, provided herein is a method of treating a tenosynovial giant cell tumor in a patient in need thereof, comprising administering to the patient 30 mg, twice a week, of a compound represented by Formula (I):

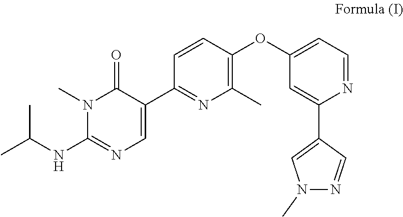 [SMILES: Cc1nc(-c2cnc(NC(C)C)n(C)c2=O)ccc1Oc1ccnc(-c2cnn(C)c2)c1]

wherein after at least 25 weeks of administering the compound, the patient achieves a partial response or a complete response as determined by Independent Radiologic Review (IRR) per tumor volume score (TVS).

In another embodiment, provided herein is a method of treating a tenosynovial giant cell tumor in a patient in need thereof, comprising administering to the patient 30 mg, twice a week, of a compound represented by:

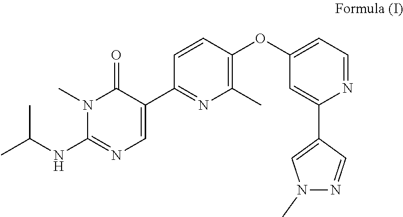 [SMILES: Cc1nc(-c2cnc(NC(C)C)n(C)c2=O)ccc1Oc1ccnc(-c2cnn(C)c2)c1]

wherein after at least 25 weeks of administering the compound, the patient has an improved active range of motion as compared to before administration of the compound to the patient.

In some embodiments, after at least 25 weeks of administering the compound, the patient achieves at least an 18% change in active range of motion as compared to before administration of the compound to the patient.

In another embodiment, provided herein is a method of treating a tenosynovial giant cell tumor in a patient in need thereof, comprising administering to the patient 30 mg, twice a week, of a compound represented by Formula (I):

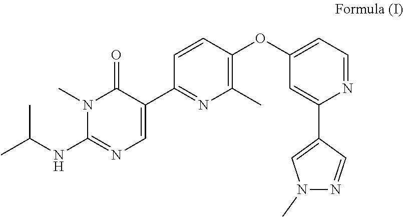 [SMILES: Cc1nc(-c2cnc(NC(C)C)n(C)c2=O)ccc1Oc1ccnc(-c2cnn(C)c2)c1]

wherein after at least 25 weeks of administering the compound, the patient achieves an improved PROMIS-PF score as compared to before administration of the compound to the patient.

In some embodiments, after at least 25 weeks of administering the compound, the patient achieves a PROMIS-PF score at least 4 points more than the patient's PROMIS-PF score before administration of the compound to the patient.

In another embodiment, provided herein is a method of treating a tenosynovial giant cell tumor in a patient in need thereof, comprising administering to the patient 30 mg, twice a week, of a compound represented by Formula (I):

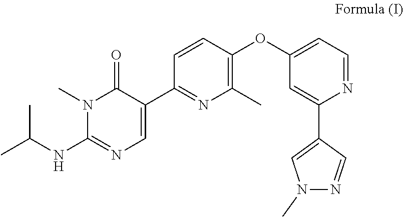 [SMILES: Cc1nc(-c2cnc(NC(C)C)n(C)c2=O)ccc1Oc1ccnc(-c2cnn(C)c2)c1]

wherein after at least 25 weeks of administering the compound, the patient achieves an improved score under the Worst Stiffness Numeric Rating Scale as compared to before administration of the compound to the patient.

In some embodiments, after at least 25 weeks of administering the compound, the patient achieves a score of, at most, −2 under the Worst Stiffness Numeric Rating Scale as compared to before administration of the compound to the patient.

In another embodiment, provided herein is a method of treating a tenosynovial giant cell tumor in a patient in need thereof, comprising administering to the patient 30 mg, twice a week, of a compound represented by Formula (I):

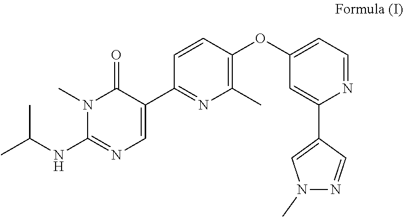 [SMILES: Cc1nc(-c2cnc(NC(C)C)n(C)c2=O)ccc1Oc1ccnc(-c2cnn(C)c2)c1]

wherein after at least 25 weeks of administering the compound, the patient achieves an improved score under the EQ-Visual Analog Scale (EQ-VAS) as compared to before administration of the compound to the patient.

In some embodiments, after at least 25 weeks of administering the compound, the patient achieves an EQ-VAS score at least 13 points more than the patient's EQ-VAS score before administration of the compound to the patient.

In another embodiment, provided herein is a method of treating a tenosynovial giant cell tumor in a patient in need thereof, comprising administering to the patient 30 mg, twice a week, of a compound represented by Formula (I):

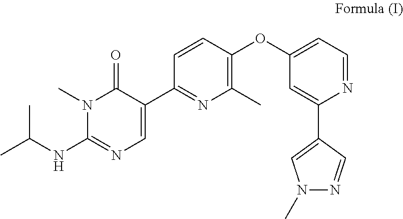 [SMILES: Cc1nc(-c2cnc(NC(C)C)n(C)c2=O)ccc1Oc1ccnc(-c2cnn(C)c2)c1]

wherein after at least 25 weeks of administering the compound, the patient achieves a greater than or equal to 30% reduction in score under the Brief Pain Inventory Numerical Rating Scale as compared to before administration of the compound to the patient.

In some embodiments, after at least 25 weeks of administering the compound, the patient sustains a less than 30% increase in narcotics use as compared to before administration of the compound to the patient.

In another embodiment, provided herein is a method of treating a patient having elevated levels of creatine phosphokinase (CPK) while being administered, for the treatment of a tenosynovial giant cell tumor, 30 mg, twice a week, of a compound represented by Formula (I):

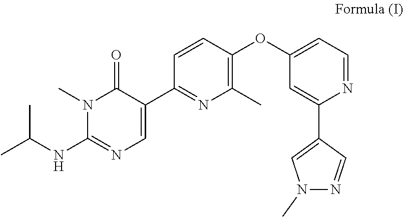 [SMILES: Cc1nc(-c2cnc(NC(C)C)n(C)c2=O)ccc1Oc1ccnc(-c2cnn(C)c2)c1]

the method comprising administering the compound to the patient in a dose that is less than 30 mg twice a week.

In some embodiments, the dose is selected from 14 mg twice a week and 20 mg twice a week. In some embodiments, the dose is 20 mg twice a week. In some embodiments, the dose is 14 mg twice a week.

In some embodiments, the tenosynovial giant cell tumor is a diffuse tenosynovial giant cell tumor. In some embodiments, the tenosynovial giant cell tumor is a localized tenosynovial giant cell tumor. In some embodiments, the patient has undergone prior surgery to treat the tenosynovial giant cell tumor. In some embodiments, the patient has been administered a prior systemic therapy for the treatment of tenosynovial giant cell tumor. In some embodiments, the systemic therapy is selected from imatinib and nilotinib. In some embodiments, the tenosynovial giant cell tumor is present in a joint in a lower limb of the patient. In some embodiments, the joint in the lower limb is selected from the group consisting of a knee, an ankle, and a hip. In some embodiments, the tenosynovial giant cell tumor is present in a joint in an upper limb of the patient. In some embodiments, the joint in the upper limb is selected from the group consisting of an elbow, a shoulder, a wrist, and a joint on the hand of the patient. In some embodiments, the patient is administered the compound represented by Formula (I) as a crystalline dihydrate form in an amount sufficient to provide 30 mg of the compound represented by Formula (I) twice a week. In some embodiments, the compound is administered to the patient for 25 weeks, 35 weeks, or 45 weeks. In some embodiments, the compound is administered to the patient for 7 months, 8 months, 9 months, 10 months, 11 months, 12 months, 13 months, 14 months, 15 months, or 16 months. In some embodiments, the compound is administered to the patient for 1 year, 2 years, 3 years, or 4 years.

In an additional embodiment, provided herein is a method of treating a tenosynovial giant cell tumor in a patient in need thereof, comprising administering to the patient 30 mg, twice a week, of a compound represented by Formula (I):

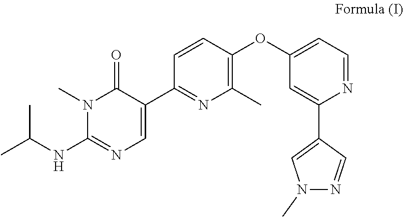 [SMILES: Cc1nc(-c2cnc(NC(C)C)n(C)c2=O)ccc1Oc1ccnc(-c2cnn(C)c2)c1]

wherein after 25 weeks of administering the compound, the patient achieves a partial response or a complete response as determined by RECIST v1.1.

In another embodiment, provided herein is a method of treating a tenosynovial giant cell tumor in a patient in need thereof, comprising administering to the patient 30 mg, twice a week, of a compound represented by Formula (I):

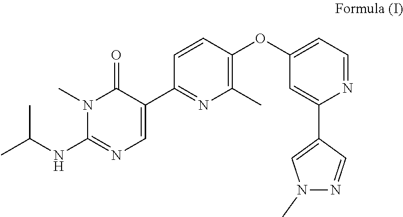 [SMILES: Cc1nc(-c2cnc(NC(C)C)n(C)c2=O)ccc1Oc1ccnc(-c2cnn(C)c2)c1]

wherein after 25 weeks of administering the compound, the patient achieves a partial response or a complete response as determined by Independent Radiologic Review (IRR) per tumor volume score (TVS.)

In another embodiment, provided herein is a method of treating a tenosynovial giant cell tumor in a patient in need thereof, comprising administering to the patient 30 mg, twice a week, of a compound represented by:

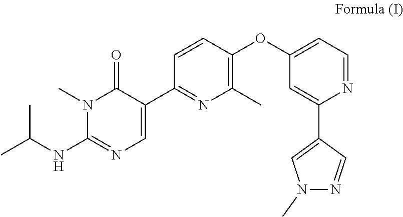 [SMILES: Cc1nc(-c2cnc(NC(C)C)n(C)c2=O)ccc1Oc1ccnc(-c2cnn(C)c2)c1]

wherein after 25 weeks of administering the compound, the patient has an improved active range of motion as compared to before administration of the compound to the patient.

In some embodiments, after 25 weeks of administering the compound, the patient achieves at least an 18% change in active range of motion as compared to before administration of the compound to the patient.

In another embodiment, provided herein is a method of treating a tenosynovial giant cell tumor in a patient in need thereof, comprising administering to the patient 30 mg, twice a week, of a compound represented by Formula (I):

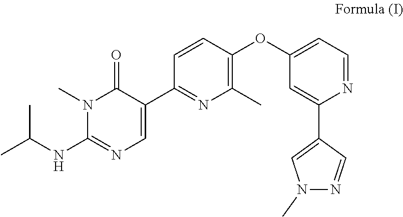 [SMILES: Cc1nc(-c2cnc(NC(C)C)n(C)c2=O)ccc1Oc1ccnc(-c2cnn(C)c2)c1]

wherein after 25 weeks of administering the compound, the patient achieves an improved PROMIS-PF score as compared to before administration of the compound to the patient.

In some embodiments, after 25 weeks of administering the compound, the patient achieves a PROMIS-PF score at least 4 points more than the patient's PROMIS-PF score before administration of the compound to the patient.

In another embodiment, provided herein is a method of treating a tenosynovial giant cell tumor in a patient in need thereof, comprising administering to the patient 30 mg, twice a week, of a compound represented by Formula (I):

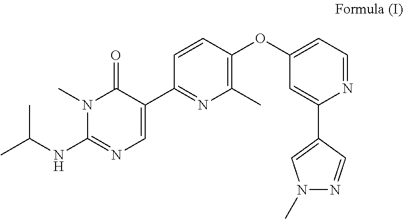 [SMILES: Cc1nc(-c2cnc(NC(C)C)n(C)c2=O)ccc1Oc1ccnc(-c2cnn(C)c2)c1]

wherein after 25 weeks of administering the compound, the patient achieves an improved score under the Worst Stiffness Numeric Rating Scale as compared to before administration of the compound to the patient.

In some embodiments, after 25 weeks of administering the compound, the patient achieves a score of, at most, −2 under the Worst Stiffness Numeric Rating Scale as compared to before administration of the compound to the patient.

In another embodiment, provided herein is a method of treating a tenosynovial giant cell tumor in a patient in need thereof, comprising administering to the patient 30 mg, twice a week, of a compound represented by Formula (I):

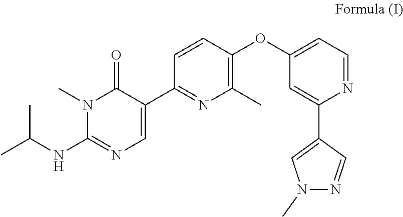 [SMILES: Cc1nc(-c2cnc(NC(C)C)n(C)c2=O)ccc1Oc1ccnc(-c2cnn(C)c2)c1]

wherein after 25 weeks of administering the compound, the patient achieves an improved score under the EQ-Visual Analog Scale (EQ-VAS) as compared to before administration of the compound to the patient.

In some embodiments, after 25 weeks of administering the compound, the patient achieves an EQ-VAS score at least 13 points more than the patient's EQ-VAS score before administration of the compound to the patient.

In another embodiment, provided herein is a method of treating a tenosynovial giant cell tumor in a patient in need thereof, comprising administering to the patient 30 mg, twice a week, of a compound represented by Formula (I):

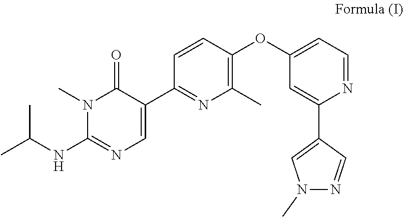 [SMILES: Cc1nc(-c2cnc(NC(C)C)n(C)c2=O)ccc1Oc1ccnc(-c2cnn(C)c2)c1]

wherein after 25 weeks of administering the compound, the patient achieves a greater than or equal to 30% reduction in score under the Brief Pain Inventory Numerical Rating Scale as compared to before administration of the compound to the patient.

In some embodiments, after 25 weeks of administering the compound, the patient sustains a less than 30% increase in narcotics use as compared to before administration of the compound to the patient.

In some embodiments, the tenosynovial giant cell tumor is a diffuse tenosynovial giant cell tumor. In some embodiments, the tenosynovial giant cell tumor is a localized tenosynovial giant cell tumor. In some embodiments, the patient has undergone prior surgery to treat the tenosynovial giant cell tumor. In some embodiments, the patient has been administered a prior systemic therapy for the treatment of tenosynovial giant cell tumor. In some embodiments, the systemic therapy is selected from imatinib and nilotinib. In some embodiments, the tenosynovial giant cell tumor is present in a joint in a lower limb of the patient. In some embodiments, the joint in the lower limb is selected from the group consisting of a knee, an ankle, and a hip. In some embodiments, the tenosynovial giant cell tumor is present in a joint in an upper limb of the patient. In some embodiments, the joint in the upper limb is selected from the group consisting of an elbow, a shoulder, a wrist, and a joint on the hand of the patient. In some embodiments, the patient is administered the compound represented by Formula (I) as a crystalline dihydrate form in an amount sufficient to provide 30 mg of the compound represented by Formula (I) twice a week. In some embodiments, the compound is administered to the patient for 25 weeks, 35 weeks, or 45 weeks. In some embodiments, the compound is administered to the patient for 7 months, 8 months, 9 months, 10 months, 11 months, 12 months, 13 months, 14 months, 15 months, or 16 months. In some embodiments, the compound is administered to the patient for 1 year, 2 years, 3 years, or 4 years.

In an additional embodiment, provided herein is a method of treating a tenosynovial giant cell tumor in a patient in need thereof, comprising administering to the patient 30 mg, twice a week, of a compound represented by Formula (I):

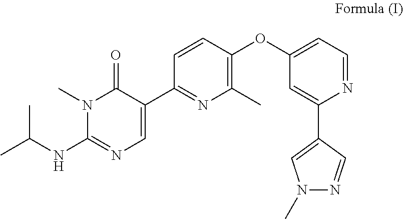 [SMILES: Cc1nc(-c2cnc(NC(C)C)n(C)c2=O)ccc1Oc1ccnc(-c2cnn(C)c2)c1]

wherein upon administering the compound to the patient for 25 weeks, the patient achieves a partial response or a complete response as determined by RECIST v1.1.

In another embodiment, provided herein is a method of treating a tenosynovial giant cell tumor in a patient in need thereof, comprising administering to the patient 30 mg, twice a week, of a compound represented by Formula (I):

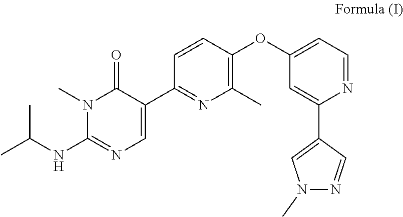 [SMILES: Cc1nc(-c2cnc(NC(C)C)n(C)c2=O)ccc1Oc1ccnc(-c2cnn(C)c2)c1]

wherein upon administering the compound to the patient for 25 weeks, the patient achieves a partial response or a complete response as determined by Independent Radiologic Review (IRR) per tumor volume score (TVS).

In another embodiment, provided herein is a method of treating a tenosynovial giant cell tumor in a patient in need thereof, comprising administering to the patient 30 mg, twice a week, of a compound represented by:

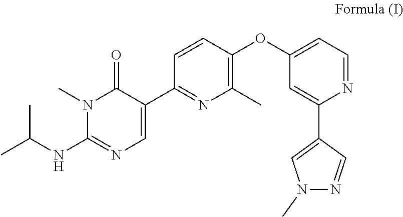 [SMILES: Cc1nc(-c2cnc(NC(C)C)n(C)c2=O)ccc1Oc1ccnc(-c2cnn(C)c2)c1]

wherein upon administering the compound to the patient for 25 weeks, the patient has an improved active range of motion as compared to before administration of the compound to the patient.

In some embodiments, upon administering the compound to the patient for 25 weeks, the patient achieves at least an 18% change in active range of motion as compared to before administration of the compound to the patient.

In another embodiment, provided herein is a method of treating a tenosynovial giant cell tumor in a patient in need thereof, comprising administering to the patient 30 mg, twice a week, of a compound represented by Formula (I):

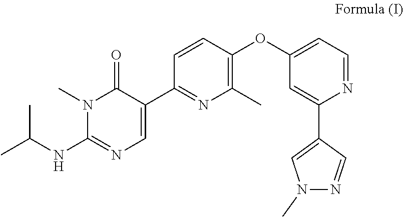 [SMILES: Cc1nc(-c2cnc(NC(C)C)n(C)c2=O)ccc1Oc1ccnc(-c2cnn(C)c2)c1]

wherein upon administering the compound to the patient for 25 weeks, the patient achieves an improved PROMIS-PF score as compared to before administration of the compound to the patient.

In some embodiments, upon administering the compound to the patient for 25 weeks, the patient achieves a PROMIS-PF score at least 4 points more than the patient's PROMIS-PF score before administration of the compound to the patient.

In another embodiment, provided herein is a method of treating a tenosynovial giant cell tumor in a patient in need thereof, comprising administering to the patient 30 mg, twice a week, of a compound represented by Formula (I):

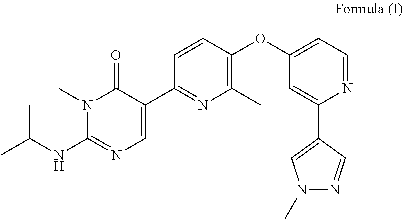 [SMILES: Cc1nc(-c2cnc(NC(C)C)n(C)c2=O)ccc1Oc1ccnc(-c2cnn(C)c2)c1]

wherein upon administering the compound to the patient for 25 weeks, the patient achieves an improved score under the Worst Stiffness Numeric Rating Scale as compared to before administration of the compound to the patient.

In some embodiments, upon administering the compound to the patient for 25 weeks, the patient achieves a score of, at most, −2 under the Worst Stiffness Numeric Rating Scale as compared to before administration of the compound to the patient.

In another embodiment, provided herein is a method of treating a tenosynovial giant cell tumor in a patient in need thereof, comprising administering to the patient 30 mg, twice a week, of a compound represented by Formula (I):

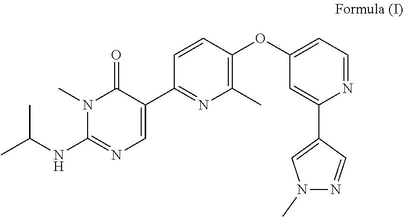 [SMILES: Cc1nc(-c2cnc(NC(C)C)n(C)c2=O)ccc1Oc1ccnc(-c2cnn(C)c2)c1]

wherein upon administering the compound to the patient for 25 weeks, the patient achieves an improved score under the EQ-Visual Analog Scale (EQ-VAS) as compared to before administration of the compound to the patient.

In some embodiments, upon administering the compound to the patient for 25 weeks, the patient achieves an EQ-VAS score at least 13 points more than the patient's EQ-VAS score before administration of the compound to the patient.

In another embodiment, provided herein is a method of treating a tenosynovial giant cell tumor in a patient in need thereof, comprising administering to the patient 30 mg, twice a week, of a compound represented by Formula (I):

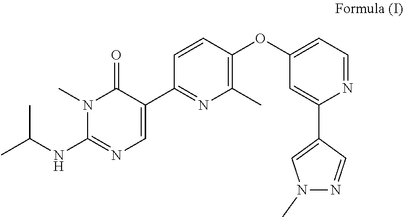 [SMILES: Cc1nc(-c2cnc(NC(C)C)n(C)c2=O)ccc1Oc1ccnc(-c2cnn(C)c2)c1]

wherein upon administering the compound to the patient for 25 weeks, the patient achieves a greater than or equal to 30% reduction in score under the Brief Pain Inventory Numerical Rating Scale as compared to before administration of the compound to the patient.

In some embodiments, upon administering the compound to the patient for 25 weeks, the patient sustains a less than 30% increase in narcotics use as compared to before administration of the compound to the patient.

In some embodiments, the compound is administered to the patient twice a week (e.g., 30 mg twice a week, 20 mg twice a week, or 14 mg twice a week), wherein, during the two-week period, the second administration of the compound to the patient is about two days, about three days, about four days, about five days, about six days, about seven days, about eight days, about nine days, about ten days, about eleven days, about twelve days, or about thirteen days after the first administration of the compound to the patient. In some embodiments, the compound is administered to the patient twice a week (e.g., 30 mg twice a week, 20 mg twice a week, or 14 mg twice a week), wherein, during the two-week period, the second administration of the compound to the patient is about four days after the first administration of the compound to the patient. In some embodiments, the compound is administered to the patient twice a week (e.g., 30 mg twice a week, 20 mg twice a week, or 14 mg twice a week), wherein, during the two-week period, the first administration of the compound to the patient is on Day 1, the second administration of the compound to the patient is on Day 5, the third administration of the compound to the patient is on Day 8, and the fourth administration of the compound to the patient is on Day 12.

In some embodiments, the tenosynovial giant cell tumor is a diffuse tenosynovial giant cell tumor. In some embodiments, the tenosynovial giant cell tumor is a localized tenosynovial giant cell tumor. In some embodiments, the patient has undergone prior surgery to treat the tenosynovial giant cell tumor. In some embodiments, the patient has been administered a prior systemic therapy for the treatment of tenosynovial giant cell tumor. In some embodiments, the systemic therapy is selected from imatinib and nilotinib. In some embodiments, the tenosynovial giant cell tumor is present in a joint in a lower limb of the patient. In some embodiments, the joint in the lower limb is selected from the group consisting of a knee, an ankle, and a hip. In some embodiments, the tenosynovial giant cell tumor is present in a joint in an upper limb of the patient. In some embodiments, the joint in the upper limb is selected from the group consisting of an elbow, a shoulder, a wrist, and a joint on the hand of the patient. In some embodiments, the patient is administered the compound represented by Formula (I) as a crystalline dihydrate form in an amount sufficient to provide 30 mg of the compound represented by Formula (I) twice a week.

As used herein and unless otherwise indicated, the term “substantially pure” when used to describe a dihydrate crystalline form of the compound represented by Formula (I) means the dihydrate crystalline form of the compound is substantially free of other solid-state or amorphous forms. In some embodiments, provided herein is a crystalline dihydrate form of the compound represented by Formula (I) which is substantially pure. In some embodiments, provided herein is a crystalline dihydrate form of the compound represented by Formula (I) that is substantially free of any other solid-state or amorphous forms, unless indicated otherwise. In some embodiments, a crystalline dihydrate form of the compound represented by Formula (I) comprises greater than about 80% by weight of the dihydrate crystalline form and less than about 20% by weight of other solid-state or amorphous forms of the compound represented by Formula (I). In some embodiments, a crystalline dihydrate form of the compound represented by Formula (I) comprises greater than about 90% by weight of the dihydrate crystalline form and less than about 10% by weight of other solid-state or amorphous forms of the compound represented by Formula (I). In some embodiments, a crystalline dihydrate form of the compound represented by Formula (I) comprises greater than about 95% by weight of the dihydrate crystalline form and less than about 5% by weight of other solid-state or amorphous forms of the compound represented by Formula (I). In some embodiments, a crystalline dihydrate form of the compound represented by Formula (I) comprises greater than about 97% by weight of the dihydrate crystalline form and less than about 3% by weight of other solid-state or amorphous forms of the compound represented by Formula (I). In some embodiments, a crystalline dihydrate form of the compound represented by Formula (I) comprises greater than about 98% by weight of the dihydrate crystalline form and less than about 2% by weight of other solid-state or amorphous forms of the compound represented by Formula (I). In some embodiments, a crystalline dihydrate form of the compound represented by Formula (I) comprises greater than about 99% by weight of the dihydrate crystalline form and less than about 1% by weight of other solid-state or amorphous forms of the compound represented by Formula (I). In some embodiments, a crystalline dihydrate form of the compound represented by Formula (I) comprises greater than about 99.5% by weight of the dihydrate crystalline form and less than about 0.5% by weight of other solid-state or amorphous forms of the compound represented by Formula (I).

As used herein, “substantially free of any other solid-state or amorphous forms” means that the solid-state form of the compound represented by Formula (I) contains about 20% or less, about 10% or less, about 5% or less, about 3% or less, about 2% or less, about 1% or less, or about 0.5% or less, of any other solid-state or amorphous form of the compound represented by Formula (I) as measured, for example, by XRPD. Thus, a dihydrate crystalline form of the compound represented by Formula (I) described herein as substantially free of any other solid-state or amorphous forms would be understood to contain greater than 80% (w/w), greater than 90% (w/w), greater than 95% (w/w), greater than 98% (w/w), about 99% or less, or greater than 99.5% (w/w) of the dihydrate crystalline form of the compound represented by Formula (I). Accordingly, in some embodiments, a crystalline dihydrate form of the compound represented by Formula (I) may contain from 0.5% to 20% (w/w), from 5% to 20% (w/w), or from 5% to 10% (w/w) of one or more other solid-state or amorphous forms of the compound represented by Formula (I).

In some embodiments, provided herein is a crystalline dihydrate form of the compound represented by Formula (I), which has a purity of greater than 80% by weight. In some embodiments, provided herein is a crystalline dihydrate form of the compound represented by Formula (I), which has a purity of greater than 90% by weight. In some embodiments, provided herein is ta crystalline dihydrate form of the compound represented by Formula (I), which has a purity of greater than 95% by weight. In some embodiments, provided herein is a crystalline dihydrate form of the compound represented by Formula (I), which has a purity of greater than 97% by weight. In some embodiments, provided herein is a crystalline dihydrate form of the compound represented by Formula (I), which has a purity of greater than 98% by weight. In some embodiments, provided herein is a crystalline dihydrate form of the compound represented by Formula (I), which has a purity of greater than 99% by weight. In some embodiments, provided herein is a crystalline dihydrate form of the compound represented by Formula (I), which has a purity of greater than 99.5% by weight.

A tenosynovial giant cell tumor in any of the present methods described herein may affect (e.g., be localized within) one or more joints of a patient. Affected joints may include an elbow, knee, hip, a joint of a hand, jaw, shoulder, wrist, and a joint of a foot. In some embodiments, the present methods described herein may affect (e.g., be localized within) any joints.

In some embodiments, the administration of the compound of Formula (I) to the patient, as described in the methods disclosed herein, is not preceded by administration of a loading dose regimen of the compound of Formula (I). In some embodiments, the administration of the compound of Formula (I) to the patient, as described in the methods disclosed herein, is not immediately preceded by administration of a loading dose regimen of the compound of Formula (I).

In one embodiment, described herein is a method of treating a tenosynovial giant cell tumor in a patient in need thereof and who is not amenable to surgical treatment of the tenosynovial giant cell tumor, comprising administering to the patient 30 mg, twice a week, of a compound represented by Formula (I):

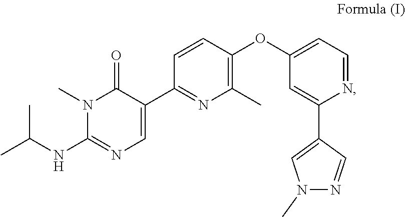 [SMILES: Cc1nc(-c2cnc(NC(C)C)n(C)c2=O)ccc1Oc1ccnc(-c2cnn(C)c2)c1]

wherein administration of the compound is not preceded by administration of a loading dose regimen of the compound to the patient. In some embodiments, described herein is a method of treating a tenosynovial giant cell tumor in a patient in need thereof and who is not amenable to surgical treatment of the tenosynovial giant cell tumor, comprising administering to the patient 30 mg, twice a week, of a compound represented by Formula (I), wherein administration of the compound is not immediately preceded by administration of a loading dose regimen of the compound to the patient.

In some embodiments, the patient does not qualify for surgery. In some embodiments, the patient is surgically ineligible (e.g., for surgical treatment of TGCT). In some embodiments, the surgical treatment is a high-risk procedure. In some embodiments, the patient has refused the surgical treatment.

In an additional embodiment, provided herein is a method of treating a tenosynovial giant cell tumor in a patient in need thereof, comprising administering to the patient 30 mg, twice a week, of a compound represented by Formula (I):

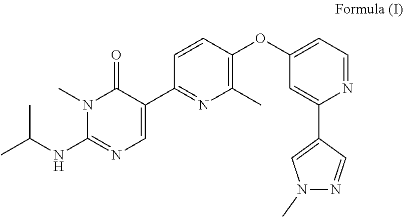 [SMILES: Cc1nc(-c2cnc(NC(C)C)n(C)c2=O)ccc1Oc1ccnc(-c2cnn(C)c2)c1]

wherein after at least 25 weeks of administering the compound, the patient achieves a partial response or a complete response as determined by RECIST v1.1, and wherein administration of the compound is not preceded by administration of a loading dose regimen of the compound to the patient. In some embodiments, provided herein is a method of treating a tenosynovial giant cell tumor in a patient in need thereof, comprising administering to the patient 30 mg, twice a week, of a compound represented by Formula (I), wherein after at least 25 weeks of administering the compound, the patient achieves a partial response or a complete response as determined by RECIST v1.1, and wherein administration of the compound is not immediately preceded by administration of a loading dose regimen of the compound to the patient.

In another embodiment, provided herein is a method of treating a tenosynovial giant cell tumor in a patient in need thereof, comprising administering to the patient 30 mg, twice a week, of a compound represented by Formula (I):

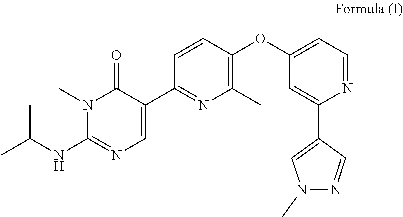 [SMILES: Cc1nc(-c2cnc(NC(C)C)n(C)c2=O)ccc1Oc1ccnc(-c2cnn(C)c2)c1]

wherein after at least 25 weeks of administering the compound, the patient achieves a partial response or a complete response as determined by Independent Radiologic Review (IRR) per tumor volume score (TVS), and wherein administration of the compound is not preceded by administration of a loading dose regimen of the compound to the patient. In some embodiments, provided herein is a method of treating a tenosynovial giant cell tumor in a patient in need thereof, comprising administering to the patient 30 mg, twice a week, of a compound represented by Formula (I), wherein after at least 25 weeks of administering the compound, the patient achieves a partial response or a complete response as determined by Independent Radiologic Review (IRR) per tumor volume score (TVS), and wherein administration of the compound is not immediately preceded by administration of a loading dose regimen of the compound to the patient.

In another embodiment, provided herein is a method of treating a tenosynovial giant cell tumor in a patient in need thereof, comprising administering to the patient 30 mg, twice a week, of a compound represented by Formula (I):

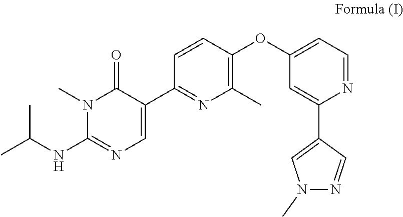 [SMILES: Cc1nc(-c2cnc(NC(C)C)n(C)c2=O)ccc1Oc1ccnc(-c2cnn(C)c2)c1]

wherein after at least 25 weeks of administering the compound, the patient has an improved active range of motion as compared to before administration of the compound to the patient, and wherein administration of the compound is not preceded by administration of a loading dose regimen of the compound to the patient. In some embodiments, provided herein is a method of treating a tenosynovial giant cell tumor in a patient in need thereof, comprising administering to the patient 30 mg, twice a week, of a compound represented by Formula (I), wherein after at least 25 weeks of administering the compound, the patient has an improved active range of motion as compared to before administration of the compound to the patient, and wherein administration of the compound is not immediately preceded by administration of a loading dose regimen of the compound to the patient.

In some embodiments, after at least 25 weeks of administering the compound, the patient achieves at least an 18% change in active range of motion as compared to before administration of the compound to the patient.

In another embodiment, provided herein is a method of treating a tenosynovial giant cell tumor in a patient in need thereof, comprising administering to the patient 30 mg, twice a week, of a compound represented by Formula (I):

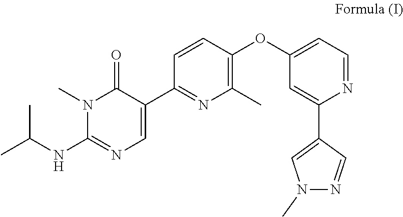 [SMILES: Cc1nc(-c2cnc(NC(C)C)n(C)c2=O)ccc1Oc1ccnc(-c2cnn(C)c2)c1]

wherein after at least 25 weeks of administering the compound, the patient achieves an improved PROMIS-PF score as compared to before administration of the compound to the patient, and wherein administration of the compound is not preceded by administration of a loading dose regimen of the compound to the patient. In some embodiments, provided herein is a method of treating a tenosynovial giant cell tumor in a patient in need thereof, comprising administering to the patient 30 mg, twice a week, of a compound represented by Formula (I): wherein after at least 25 weeks of administering the compound, the patient achieves an improved PROMIS-PF score as compared to before administration of the compound to the patient, and wherein administration of the compound is not immediately preceded by administration of a loading dose regimen of the compound to the patient.

In some embodiments, after at least 25 weeks of administering the compound, the patient achieves a PROMIS-PF score at least 4 points more than the patient's PROMIS-PF score before administration of the compound to the patient.

In another embodiment, provided herein is a method of treating a tenosynovial giant cell tumor in a patient in need thereof, comprising administering to the patient 30 mg, twice a week, of a compound represented by Formula (I):

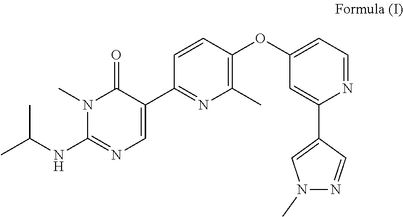 [SMILES: Cc1nc(-c2cnc(NC(C)C)n(C)c2=O)ccc1Oc1ccnc(-c2cnn(C)c2)c1]

wherein after at least 25 weeks of administering the compound, the patient achieves an improved score under the Worst Stiffness Numeric Rating Scale as compared to before administration of the compound to the patient, and wherein administration of the compound is not preceded by administration of a loading dose regimen of the compound to the patient. In some embodiments, provided herein is a method of treating a tenosynovial giant cell tumor in a patient in need thereof, comprising administering to the patient 30 mg, twice a week, of a compound represented by Formula (I), wherein after at least 25 weeks of administering the compound, the patient achieves an improved score under the Worst Stiffness Numeric Rating Scale as compared to before administration of the compound to the patient, and wherein administration of the compound is not immediately preceded by administration of a loading dose regimen of the compound to the patient.

In some embodiments, after at least 25 weeks of administering the compound, the patient achieves a score of, at most, −2 under the Worst Stiffness Numeric Rating Scale as compared to before administration of the compound to the patient.

In another embodiment, provided herein is a method of treating a tenosynovial giant cell tumor in a patient in need thereof, comprising administering to the patient 30 mg, twice a week, of a compound represented by Formula (I):

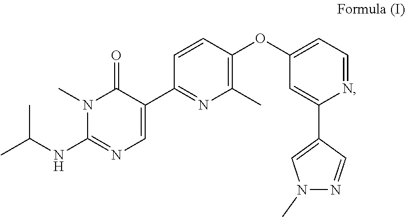 [SMILES: Cc1nc(-c2cnc(NC(C)C)n(C)c2=O)ccc1Oc1ccnc(-c2cnn(C)c2)c1]

wherein after at least 25 weeks of administering the compound, the patient achieves an improved score under the EQ-Visual Analog Scale (EQ-VAS) as compared to before administration of the compound to the patient, and wherein administration of the compound is not preceded by administration of a loading dose regimen of the compound to the patient. In some embodiments, provided herein is a method of treating a tenosynovial giant cell tumor in a patient in need thereof, comprising administering to the patient 30 mg, twice a week, of a compound represented by Formula (I), wherein after at least 25 weeks of administering the compound, the patient achieves an improved score under the EQ-Visual Analog Scale (EQ-VAS) as compared to before administration of the compound to the patient, and wherein administration of the compound is not immediately preceded by administration of a loading dose regimen of the compound to the patient.

In some embodiments, after at least 25 weeks of administering the compound, the patient achieves an EQ-VAS score at least 13 points more than the patient's EQ-VAS score before administration of the compound to the patient.

In another embodiment, provided herein is a method of treating a tenosynovial giant cell tumor in a patient in need thereof, comprising administering to the patient 30 mg, twice a week, of a compound represented by Formula (I):

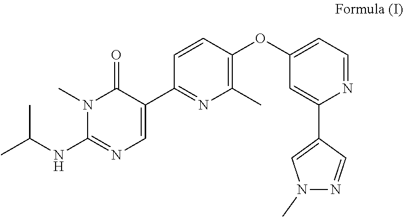 [SMILES: Cc1nc(-c2cnc(NC(C)C)n(C)c2=O)ccc1Oc1ccnc(-c2cnn(C)c2)c1]

wherein after at least 25 weeks of administering the compound, the patient achieves a greater than or equal to 30% reduction in score under the Brief Pain Inventory Numerical Rating Scale as compared to before administration of the compound to the patient, and wherein administration of the compound is not preceded by administration of a loading dose regimen of the compound to the patient. In some embodiments, provided herein is a method of treating a tenosynovial giant cell tumor in a patient in need thereof, comprising administering to the patient 30 mg, twice a week, of a compound represented by Formula (I), wherein after at least 25 weeks of administering the compound, the patient achieves a greater than or equal to 30% reduction in score under the Brief Pain Inventory Numerical Rating Scale as compared to before administration of the compound to the patient, and wherein administration of the compound is not immediately preceded by administration of a loading dose regimen of the compound to the patient.

In some embodiments, after at least 25 weeks of administering the compound, the patient sustains a less than 30% increase in narcotics use as compared to before administration of the compound to the patient.

In another embodiment, provided herein is a method of treating a patient having elevated levels of creatine phosphokinase (CPK) while being administered, for the treatment of a tenosynovial giant cell tumor, 30 mg, twice a week, of a compound represented by Formula (I):

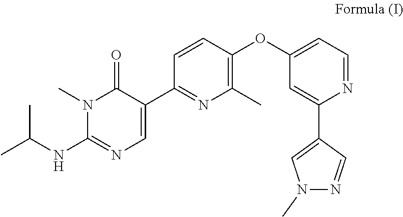 [SMILES: Cc1nc(-c2cnc(NC(C)C)n(C)c2=O)ccc1Oc1ccnc(-c2cnn(C)c2)c1]

the method comprising administering the compound to the patient in a dose that is less than 30 mg twice a week, wherein administration of the compound is not preceded by administration of a loading dose regimen of the compound to the patient. In some embodiments, provided herein is a method of treating a patient having elevated levels of creatine phosphokinase (CPK) while being administered, for the treatment of a tenosynovial giant cell tumor, 30 mg, twice a week, of a compound represented by Formula (I), the method comprising administering the compound to the patient in a dose that is less than 30 mg twice a week, wherein administration of the compound is not immediately preceded by administration of a loading dose regimen of the compound to the patient.

In some embodiments, the dose is selected from 14 mg twice a week and 20 mg twice a week. In some embodiments, the dose is 20 mg twice a week. In some embodiments, the dose is 14 mg twice a week.

In some embodiments, the tenosynovial giant cell tumor is a diffuse tenosynovial giant cell tumor. In some embodiments, the tenosynovial giant cell tumor is a localized tenosynovial giant cell tumor. In some embodiments, the patient has undergone prior surgery to treat the tenosynovial giant cell tumor. In some embodiments, the patient has been administered a prior systemic therapy for the treatment of tenosynovial giant cell tumor. In some embodiments, the systemic therapy is selected from imatinib and nilotinib. In some embodiments, the tenosynovial giant cell tumor is present in a joint in a lower limb of the patient. In some embodiments, the joint in the lower limb is selected from the group consisting of a knee, an ankle, and a hip. In some embodiments, the tenosynovial giant cell tumor is present in a joint in an upper limb of the patient. In some embodiments, the joint in the upper limb is selected from the group consisting of an elbow, a shoulder, a wrist, and a joint on the hand of the patient. In some embodiments, the patient is administered the compound represented by Formula (I) as a crystalline dihydrate form in an amount sufficient to provide 30 mg of the compound represented by Formula (I) twice a week. In some embodiments, the compound is administered to the patient for 25 weeks, 35 weeks, or 45 weeks. In some embodiments, the compound is administered to the patient for 7 months, 8 months, 9 months, 10 months, 11 months, 12 months, 13 months, 14 months, 15 months, or 16 months. In some embodiments, the compound is administered to the patient for 1 year, 2 years, 3 years, or 4 years.

In an additional embodiment, provided herein is a method of treating a tenosynovial giant cell tumor in a patient in need thereof, comprising administering to the patient 30 mg, twice a week, of a compound represented by Formula (I):

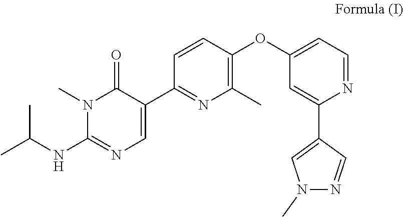 [SMILES: Cc1nc(-c2cnc(NC(C)C)n(C)c2=O)ccc1Oc1ccnc(-c2cnn(C)c2)c1]

wherein after 25 weeks of administering the compound, the patient achieves a partial response or a complete response as determined by RECIST v1.1, and wherein administration of the compound is not preceded by administration of a loading dose regimen of the compound to the patient. In some embodiments, provided herein is a method of treating a tenosynovial giant cell tumor in a patient in need thereof, comprising administering to the patient 30 mg, twice a week, of a compound represented by Formula (I), wherein after 25 weeks of administering the compound, the patient achieves a partial response or a complete response as determined by RECIST v1.1, and wherein administration of the compound is not immediately preceded by administration of a loading dose regimen of the compound to the patient.

In another embodiment, provided herein is a method of treating a tenosynovial giant cell tumor in a patient in need thereof, comprising administering to the patient 30 mg, twice a week, of a compound represented by Formula (I):

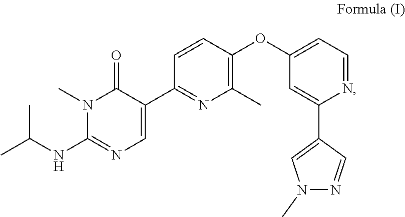 [SMILES: Cc1nc(-c2cnc(NC(C)C)n(C)c2=O)ccc1Oc1ccnc(-c2cnn(C)c2)c1]

wherein after 25 weeks of administering the compound, the patient achieves a partial response or a complete response as determined by Independent Radiologic Review (IRR) per tumor volume score (TVS), and wherein administration of the compound is not preceded by administration of a loading dose regimen of the compound to the patient. In some embodiments, provided herein is a method of treating a tenosynovial giant cell tumor in a patient in need thereof, comprising administering to the patient 30 mg, twice a week, of a compound represented by Formula (I), wherein after 25 weeks of administering the compound, the patient achieves a partial response or a complete response as determined by Independent Radiologic Review (IRR) per tumor volume score (TVS), and wherein administration of the compound is not immediately preceded by administration of a loading dose regimen of the compound to the patient.

In another embodiment, provided herein is a method of treating a tenosynovial giant cell tumor in a patient in need thereof, comprising administering to the patient 30 mg, twice a week, of a compound represented by:

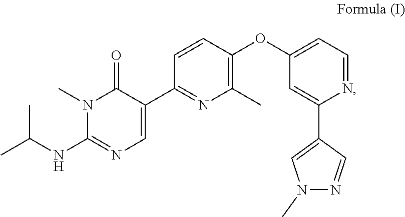 [SMILES: Cc1nc(-c2cnc(NC(C)C)n(C)c2=O)ccc1Oc1ccnc(-c2cnn(C)c2)c1]

wherein after 25 weeks of administering the compound, the patient has an improved active range of motion as compared to before administration of the compound to the patient, and wherein administration of the compound is not preceded by administration of a loading dose regimen of the compound to the patient. In some embodiments, provided herein is a method of treating a tenosynovial giant cell tumor in a patient in need thereof, comprising administering to the patient 30 mg, twice a week, of a compound represented by Formula (I), wherein after 25 weeks of administering the compound, the patient has an improved active range of motion as compared to before administration of the compound to the patient, and wherein administration of the compound is not immediately preceded by administration of a loading dose regimen of the compound to the patient.

In some embodiments, after 25 weeks of administering the compound, the patient achieves at least an 18% change in active range of motion as compared to before administration of the compound to the patient.

In another embodiment, provided herein is a method of treating a tenosynovial giant cell tumor in a patient in need thereof, comprising administering to the patient 30 mg, twice a week, of a compound represented by Formula (I):

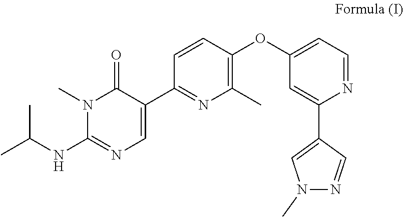 [SMILES: Cc1nc(-c2cnc(NC(C)C)n(C)c2=O)ccc1Oc1ccnc(-c2cnn(C)c2)c1]

wherein after 25 weeks of administering the compound, the patient achieves an improved PROMIS-PF score as compared to before administration of the compound to the patient, and wherein administration of the compound is not preceded by administration of a loading dose regimen of the compound to the patient. In some embodiments, provided herein is a method of treating a tenosynovial giant cell tumor in a patient in need thereof, comprising administering to the patient 30 mg, twice a week, of a compound represented by Formula (I), wherein after 25 weeks of administering the compound, the patient achieves an improved PROMIS-PF score as compared to before administration of the compound to the patient, and wherein administration of the compound is not immediately preceded by administration of a loading dose regimen of the compound to the patient.

In some embodiments, after 25 weeks of administering the compound, the patient achieves a PROMIS-PF score at least 4 points more than the patient's PROMIS-PF score before administration of the compound to the patient.

In another embodiment, provided herein is a method of treating a tenosynovial giant cell tumor in a patient in need thereof, comprising administering to the patient 30 mg, twice a week, of a compound represented by Formula (I):

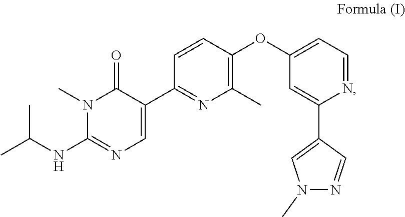 [SMILES: Cc1nc(-c2cnc(NC(C)C)n(C)c2=O)ccc1Oc1ccnc(-c2cnn(C)c2)c1]

wherein after 25 weeks of administering the compound, the patient achieves an improved score under the Worst Stiffness Numeric Rating Scale as compared to before administration of the compound to the patient, and wherein administration of the compound is not preceded by administration of a loading dose regimen of the compound to the patient. In some embodiments, provided herein is a method of treating a tenosynovial giant cell tumor in a patient in need thereof, comprising administering to the patient 30 mg, twice a week, of a compound represented by Formula (I), wherein after 25 weeks of administering the compound, the patient achieves an improved score under the Worst Stiffness Numeric Rating Scale as compared to before administration of the compound to the patient, and wherein administration of the compound is not immediately preceded by administration of a loading dose regimen of the compound to the patient.

In some embodiments, after 25 weeks of administering the compound, the patient achieves a score of, at most, −2 under the Worst Stiffness Numeric Rating Scale as compared to before administration of the compound to the patient.

In another embodiment, provided herein is a method of treating a tenosynovial giant cell tumor in a patient in need thereof, comprising administering to the patient 30 mg, twice a week, of a compound represented by Formula (I):

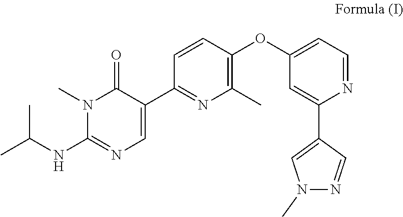 [SMILES: Cc1nc(-c2cnc(NC(C)C)n(C)c2=O)ccc1Oc1ccnc(-c2cnn(C)c2)c1]

wherein after 25 weeks of administering the compound, the patient achieves an improved score under the EQ-Visual Analog Scale (EQ-VAS) as compared to before administration of the compound to the patient, and wherein administration of the compound is not preceded by administration of a loading dose regimen of the compound to the patient. In some embodiments, provided herein is a method of treating a tenosynovial giant cell tumor in a patient in need thereof, comprising administering to the patient 30 mg, twice a week, of a compound represented by Formula (I), wherein after 25 weeks of administering the compound, the patient achieves an improved score under the EQ-Visual Analog Scale (EQ-VAS) as compared to before administration of the compound to the patient, and wherein administration of the compound is not immediately preceded by administration of a loading dose regimen of the compound to the patient.

In some embodiments, after 25 weeks of administering the compound, the patient achieves an EQ-VAS score at least 13 points more than the patient's EQ-VAS score before administration of the compound to the patient.

In another embodiment, provided herein is a method of treating a tenosynovial giant cell tumor in a patient in need thereof, comprising administering to the patient 30 mg, twice a week, of a compound represented by Formula (I):

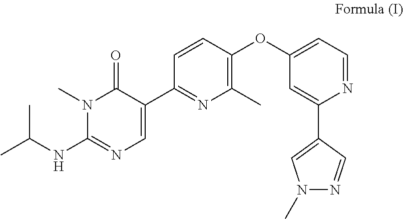 [SMILES: Cc1nc(-c2cnc(NC(C)C)n(C)c2=O)ccc1Oc1ccnc(-c2cnn(C)c2)c1]

wherein after 25 weeks of administering the compound, the patient achieves a greater than or equal to 30% reduction in score under the Brief Pain Inventory Numerical Rating Scale as compared to before administration of the compound to the patient, and wherein administration of the compound is not preceded by administration of a loading dose regimen of the compound to the patient. In some embodiments, provided herein is a method of treating a tenosynovial giant cell tumor in a patient in need thereof, comprising administering to the patient 30 mg, twice a week, of a compound represented by Formula (I), wherein after 25 weeks of administering the compound, the patient achieves a greater than or equal to 30% reduction in score under the Brief Pain Inventory Numerical Rating Scale as compared to before administration of the compound to the patient, and wherein administration of the compound is not immediately preceded by administration of a loading dose regimen of the compound to the patient.

In some embodiments, after 25 weeks of administering the compound, the patient sustains a less than 30% increase in narcotics use as compared to before administration of the compound to the patient.

In some embodiments, the dose is selected from 14 mg twice a week and 20 mg twice a week. In some embodiments, the dose is 20 mg twice a week. In some embodiments, the dose is 14 mg twice a week.

Patients being administered the compound of Formula (I) may or may not be administered another active ingredient (e.g., a breast cancer resistance protein (BCRP) substrate or a P-glycoprotein (P-gp) substrate) to treat an additional disease in the patient. In some embodiments, the additional disease is selected from the group consisting of hypercholesteremia, diabetes (e.g., diabetes mellitus), hypertension, deep vein thrombosis, and depression. In some embodiments, the patient is not administered a breast cancer resistance protein (BCRP) substrate while being administered the compound of Formula (I). In some embodiments, the patient is not administered a breast cancer resistance protein (BCRP) substrate before administering the compound of Formula (I). In some embodiments, the patient is not administered a breast cancer resistance protein (BCRP) substrate within four hours of being administered the compound of Formula (I). In some embodiments, the BCRP substrate is selected from the group consisting of a statin, a sulfonylurea (e.g., glyburide), a tyrosine kinase inhibitor, and an anticoagulant. In some embodiments, the patient is not administered a P-glycoprotein (P-gp) substrate while being administered the compound of Formula (I). In some embodiments, the patient is not administered a P-glycoprotein (P-gp) substrate before administering the compound of Formula (I). In some embodiments, the patient is not administered a P-glycoprotein (P-gp) substrate within two hours of being administered the compound of Formula (I). In some embodiments, the P-gp substrate is selected from the group consisting of an anti-hypertensive drug (e.g., calcium channel blockers or an angiotensin II receptor blocker (ARB)), an anti-viral drug, and a tri-cyclic antidepressant.

In some embodiments, the tenosynovial giant cell tumor is a diffuse tenosynovial giant cell tumor. In some embodiments, the tenosynovial giant cell tumor is a localized tenosynovial giant cell tumor. In some embodiments, the patient has undergone prior surgery to treat the tenosynovial giant cell tumor. In some embodiments, the patient has been administered a prior systemic therapy for the treatment of tenosynovial giant cell tumor. In some embodiments, the systemic therapy is selected from imatinib and nilotinib. In some embodiments, the tenosynovial giant cell tumor is present in a joint in a lower limb of the patient. In some embodiments, the joint in the lower limb is selected from the group consisting of a knee, an ankle, and a hip. In some embodiments, the tenosynovial giant cell tumor is present in a joint in an upper limb of the patient. In some embodiments, the joint in the upper limb is selected from the group consisting of an elbow, a shoulder, a wrist, and a joint on the hand of the patient. In some embodiments, the patient is administered the compound represented by Formula (I) as a crystalline dihydrate form in an amount sufficient to provide 30 mg of the compound represented by Formula (I) twice a week. In some embodiments, the compound is administered to the patient for 25 weeks, 35 weeks, or 45 weeks. In some embodiments, the compound is administered to the patient for 7 months, 8 months, 9 months, 10 months, 11 months, 12 months, 13 months, 14 months, 15 months, or 16 months. In some embodiments, the compound is administered to the patient for 1 year, 2 years, 3 years, or 4 years.

In an additional embodiment, provided herein is a method of treating a tenosynovial giant cell tumor in a patient in need thereof, comprising administering to the patient 30 mg, twice a week, of a compound represented by Formula (I):

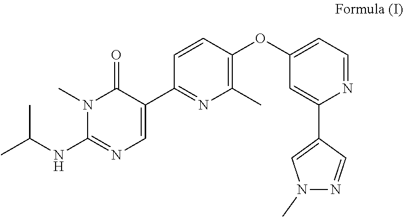 [SMILES: Cc1nc(-c2cnc(NC(C)C)n(C)c2=O)ccc1Oc1ccnc(-c2cnn(C)c2)c1]

wherein upon administering the compound to the patient for 25 weeks, the patient achieves a partial response or a complete response as determined by RECIST v1.1, and wherein administration of the compound is not preceded by administration of a loading dose regimen of the compound to the patient. In some embodiments, provided herein is a method of treating a tenosynovial giant cell tumor in a patient in need thereof, comprising administering to the patient 30 mg, twice a week, of a compound represented by Formula (I), wherein upon administering the compound to the patient for 25 weeks, the patient achieves a partial response or a complete response as determined by RECIST v1.1, and wherein administration of the compound is not immediately preceded by administration of a loading dose regimen of the compound to the patient.

In another embodiment, provided herein is a method of treating a tenosynovial giant cell tumor in a patient in need thereof, comprising administering to the patient 30 mg, twice a week, of a compound represented by Formula (I):

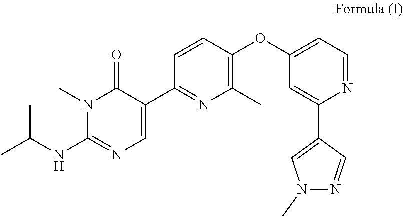 [SMILES: Cc1nc(-c2cnc(NC(C)C)n(C)c2=O)ccc1Oc1ccnc(-c2cnn(C)c2)c1]

wherein upon administering the compound to the patient for 25 weeks, the patient achieves a partial response or a complete response as determined by Independent Radiologic Review (IRR) per tumor volume score (TVS), and wherein administration of the compound is not preceded by administration of a loading dose regimen of the compound to the patient. In some embodiments, provided herein is a method of treating a tenosynovial giant cell tumor in a patient in need thereof, comprising administering to the patient 30 mg, twice a week, of a compound represented by Formula (I), wherein upon administering the compound to the patient for 25 weeks, the patient achieves a partial response or a complete response as determined by Independent Radiologic Review (IRR) per tumor volume score (TVS), and wherein administration of the compound is not immediately preceded by administration of a loading dose regimen of the compound to the patient.

In another embodiment, provided herein is a method of treating a tenosynovial giant cell tumor in a patient in need thereof, comprising administering to the patient 30 mg, twice a week, of a compound represented by Formula (I):

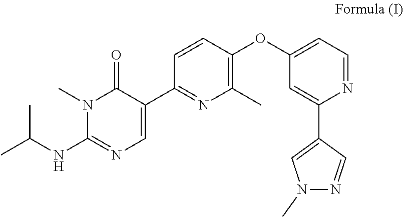 [SMILES: Cc1nc(-c2cnc(NC(C)C)n(C)c2=O)ccc1Oc1ccnc(-c2cnn(C)c2)c1]

wherein upon administering the compound to the patient for 25 weeks, the patient has an improved active range of motion as compared to before administration of the compound to the patient, and wherein administration of the compound is not preceded by administration of a loading dose regimen of the compound to the patient. In some embodiments, provided herein is a method of treating a tenosynovial giant cell tumor in a patient in need thereof, comprising administering to the patient 30 mg, twice a week, of a compound represented by Formula (I), wherein upon administering the compound to the patient for 25 weeks, the patient has an improved active range of motion as compared to before administration of the compound to the patient, and wherein administration of the compound is not immediately preceded by administration of a loading dose regimen of the compound to the patient.

In some embodiments, upon administering the compound to the patient for 25 weeks, the patient achieves at least an 18% change in active range of motion as compared to before administration of the compound to the patient.

In another embodiment, provided herein is a method of treating a tenosynovial giant cell tumor in a patient in need thereof, comprising administering to the patient 30 mg, twice a week, of a compound represented by Formula (I):

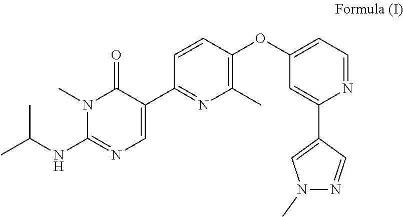 [SMILES: Cc1nc(-c2cnc(NC(C)C)n(C)c2=O)ccc1Oc1ccnc(-c2cnn(C)c2)c1]

wherein upon administering the compound to the patient for 25 weeks, the patient achieves an improved PROMIS-PF score as compared to before administration of the compound to the patient, and wherein administration of the compound is not preceded by administration of a loading dose regimen of the compound to the patient. In some embodiments, provided herein is a method of treating a tenosynovial giant cell tumor in a patient in need thereof, comprising administering to the patient 30 mg, twice a week, of a compound represented by Formula (I), wherein upon administering the compound to the patient for 25 weeks, the patient achieves an improved PROMIS-PF score as compared to before administration of the compound to the patient, and wherein administration of the compound is not immediately preceded by administration of a loading dose regimen of the compound to the patient.

In some embodiments, upon administering the compound to the patient for 25 weeks, the patient achieves a PROMIS-PF score at least 4 points more than the patient's PROMIS-PF score before administration of the compound to the patient.

In another embodiment, provided herein is a method of treating a tenosynovial giant cell tumor in a patient in need thereof, comprising administering to the patient 30 mg, twice a week, of a compound represented by Formula (I):

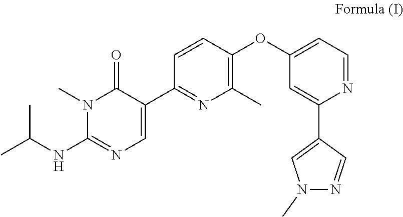 [SMILES: Cc1nc(-c2cnc(NC(C)C)n(C)c2=O)ccc1Oc1ccnc(-c2cnn(C)c2)c1]

wherein upon administering the compound to the patient for 25 weeks, the patient achieves an improved score under the Worst Stiffness Numeric Rating Scale as compared to before administration of the compound to the patient, and wherein administration of the compound is not preceded by administration of a loading dose regimen of the compound to the patient. In some embodiments, provided herein is a method of treating a tenosynovial giant cell tumor in a patient in need thereof, comprising administering to the patient 30 mg, twice a week, of a compound represented by Formula (I), wherein upon administering the compound to the patient for 25 weeks, the patient achieves an improved score under the Worst Stiffness Numeric Rating Scale as compared to before administration of the compound to the patient, and wherein administration of the compound is not immediately preceded by administration of a loading dose regimen of the compound to the patient.

In some embodiments, upon administering the compound to the patient for 25 weeks, the patient achieves a score of, at most, −2 under the Worst Stiffness Numeric Rating Scale as compared to before administration of the compound to the patient.

In another embodiment, provided herein is a method of treating a tenosynovial giant cell tumor in a patient in need thereof, comprising administering to the patient 30 mg, twice a week, of a compound represented by Formula (I):

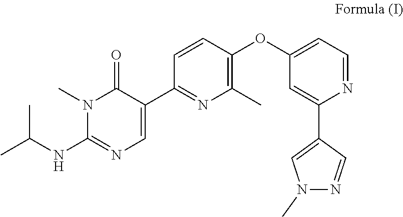 [SMILES: Cc1nc(-c2cnc(NC(C)C)n(C)c2=O)ccc1Oc1ccnc(-c2cnn(C)c2)c1]

wherein upon administering the compound to the patient for 25 weeks, the patient achieves an improved score under the EQ-Visual Analog Scale (EQ-VAS) as compared to before administration of the compound to the patient, and wherein administration of the compound is not preceded by administration of a loading dose regimen of the compound to the patient. In some embodiments, provided herein is a method of treating a tenosynovial giant cell tumor in a patient in need thereof, comprising administering to the patient 30 mg, twice a week, of a compound represented by Formula (I), wherein upon administering the compound to the patient for 25 weeks, the patient achieves an improved score under the EQ-Visual Analog Scale (EQ-VAS) as compared to before administration of the compound to the patient, and wherein administration of the compound is not immediately preceded by administration of a loading dose regimen of the compound to the patient.

In some embodiments, upon administering the compound to the patient for 25 weeks, the patient achieves an EQ-VAS score at least 13 points more than the patient's EQ-VAS score before administration of the compound to the patient.

In another embodiment, provided herein is a method of treating a tenosynovial giant cell tumor in a patient in need thereof, comprising administering to the patient 30 mg, twice a week, of a compound represented by Formula (I):

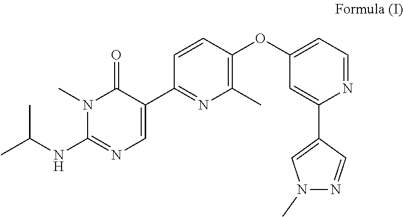 [SMILES: Cc1nc(-c2cnc(NC(C)C)n(C)c2=O)ccc1Oc1ccnc(-c2cnn(C)c2)c1]

wherein upon administering the compound to the patient for 25 weeks, the patient achieves a greater than or equal to 30% reduction in score under the Brief Pain Inventory Numerical Rating Scale as compared to before administration of the compound to the patient, and wherein administration of the compound is not preceded by administration of a loading dose regimen of the compound to the patient. In some embodiments, provided herein is a method of treating a tenosynovial giant cell tumor in a patient in need thereof, comprising administering to the patient 30 mg, twice a week, of a compound represented by Formula (I), wherein upon administering the compound to the patient for 25 weeks, the patient achieves a greater than or equal to 30% reduction in score under the Brief Pain Inventory Numerical Rating Scale as compared to before administration of the compound to the patient, and wherein administration of the compound is not immediately preceded by administration of a loading dose regimen of the compound to the patient.

In some embodiments, upon administering the compound to the patient for 25 weeks, the patient sustains a less than 30% increase in narcotics use as compared to before administration of the compound to the patient.

In some embodiments, the dose is selected from 14 mg twice a week and 20 mg twice a week. In some embodiments, the dose is 20 mg twice a week. In some embodiments, the dose is 14 mg twice a week.

In some embodiments, the compound is administered to the patient twice a week (e.g., 30 mg twice a week, 20 mg twice a week, or 14 mg twice a week), wherein, during the two-week period, the second administration of the compound to the patient is about two days, about three days, about four days, about five days, about six days, about seven days, about eight days, about nine days, about ten days, about eleven days, about twelve days, or about thirteen days after the first administration of the compound to the patient. In some embodiments, the compound is administered to the patient twice a week (e.g., 30 mg twice a week, 20 mg twice a week, or 14 mg twice a week), wherein, during the two-week period, the second administration of the compound to the patient is about four days after the first administration of the compound to the patient. In some embodiments, the compound is administered to the patient twice a week (e.g., 30 mg twice a week, 20 mg twice a week, or 14 mg twice a week), wherein, during the two-week period, the first administration of the compound to the patient is on Day 1, the second administration of the compound to the patient is on Day 5, the third administration of the compound to the patient is on Day 8, and the fourth administration of the compound to the patient is on Day 12.

In some embodiments, the tenosynovial giant cell tumor is a diffuse tenosynovial giant cell tumor. In some embodiments, the tenosynovial giant cell tumor is a localized tenosynovial giant cell tumor. In some embodiments, the patient has undergone prior surgery to treat the tenosynovial giant cell tumor. In some embodiments, the patient has been administered a prior systemic therapy for the treatment of tenosynovial giant cell tumor. In some embodiments, the systemic therapy is selected from imatinib and nilotinib. In some embodiments, the tenosynovial giant cell tumor is present in a joint in a lower limb of the patient. In some embodiments, the joint in the lower limb is selected from the group consisting of a knee, an ankle, and a hip. In some embodiments, the tenosynovial giant cell tumor is present in a joint in an upper limb of the patient. In some embodiments, the joint in the upper limb is selected from the group consisting of an elbow, a shoulder, a wrist, and a joint on the hand of the patient. In some embodiments, the patient is administered the compound represented by Formula (I) as a crystalline dihydrate form in an amount sufficient to provide 30 mg of the compound represented by Formula (I) twice a week.

As used herein and unless otherwise indicated, the term “substantially pure” when used to describe a dihydrate crystalline form of the compound represented by Formula (I) means the dihydrate crystalline form of the compound is substantially free of other solid-state or amorphous forms. In some embodiments, provided herein is a crystalline dihydrate form of the compound represented by Formula (I) which is substantially pure. In some embodiments, provided herein is a crystalline dihydrate form of the compound represented by Formula (I) that is substantially free of any other solid-state or amorphous forms, unless indicated otherwise. In some embodiments, a crystalline dihydrate form of the compound represented by Formula (I) comprises greater than about 80% by weight of the dihydrate crystalline form and less than about 20% by weight of other solid-state or amorphous forms of the compound represented by Formula (I). In some embodiments, a crystalline dihydrate form of the compound represented by Formula (I) comprises greater than about 90% by weight of the dihydrate crystalline form and less than about 10% by weight of other solid-state or amorphous forms of the compound represented by Formula (I). In some embodiments, a crystalline dihydrate form of the compound represented by Formula (I) comprises greater than about 95% by weight of the dihydrate crystalline form and less than about 5% by weight of other solid-state or amorphous forms of the compound represented by Formula (I). In some embodiments, a crystalline dihydrate form of the compound represented by Formula (I) comprises greater than about 97% by weight of the dihydrate crystalline form and less than about 3% by weight of other solid-state or amorphous forms of the compound represented by Formula (I). In some embodiments, a crystalline dihydrate form of the compound represented by Formula (I) comprises greater than about 98% by weight of the dihydrate crystalline form and less than about 2% by weight of other solid-state or amorphous forms of the compound represented by Formula (I). In some embodiments, a crystalline dihydrate form of the compound represented by Formula (I) comprises greater than about 99% by weight of the dihydrate crystalline form and less than about 1% by weight of other solid-state or amorphous forms of the compound represented by Formula (I). In some embodiments, a crystalline dihydrate form of the compound represented by Formula (I) comprises greater than about 99.5% by weight of the dihydrate crystalline form and less than about 0.5% by weight of other solid-state or amorphous forms of the compound represented by Formula (I).

As used herein, “substantially free of any other solid-state or amorphous forms” means that the solid-state form of the compound represented by Formula (I) contains about 20% or less, about 10% or less, about 5% or less, about 3% or less, about 2% or less, about 1% or less, or about 0.5% or less, of any other solid-state or amorphous form of the compound represented by Formula (I) as measured, for example, by XRPD. Thus, a dihydrate crystalline form of the compound represented by Formula (I) described herein as substantially free of any other solid-state or amorphous forms would be understood to contain greater than 80% (w/w), greater than 90% (w/w), greater than 95% (w/w), greater than 98% (w/w), about 99% or less, or greater than 99.5% (w/w) of the dihydrate crystalline form of the compound represented by Formula (I). Accordingly, in some embodiments, a crystalline dihydrate form of the compound represented by Formula (I) may contain from 0.5% to 20% (w/w), from 5% to 20% (w/w), or from 5% to 10% (w/w) of one or more other solid-state or amorphous forms of the compound represented by Formula (I).

In some embodiments, provided herein is a crystalline dihydrate form of the compound represented by Formula (I), which has a purity of greater than 80% by weight. In some embodiments, provided herein is a crystalline dihydrate form of the compound represented by Formula (I), which has a purity of greater than 90% by weight. In some embodiments, provided herein is ta crystalline dihydrate form of the compound represented by Formula (I), which has a purity of greater than 95% by weight. In some embodiments, provided herein is a crystalline dihydrate form of the compound represented by Formula (I), which has a purity of greater than 97% by weight. In some embodiments, provided herein is a crystalline dihydrate form of the compound represented by Formula (I), which has a purity of greater than 98% by weight. In some embodiments, provided herein is a crystalline dihydrate form of the compound represented by Formula (I), which has a purity of greater than 99% by weight. In some embodiments, provided herein is a crystalline dihydrate form of the compound represented by Formula (I), which has a purity of greater than 99.5% by weight.

A tenosynovial giant cell tumor in any of the present methods described herein may affect (e.g., be localized within) one or more joints of a patient. Affected joints may include an elbow, knee, hip, a joint of a hand, jaw, shoulder, wrist, and a joint of a foot. In some embodiments, the present methods described herein may affect (e.g., be localized within) any joints.

### Dose Interruption and Modification

Dose modifications may be made in the methods of administering the compound represented by Formula (I) described herein as a result of adverse events experienced by the patient. In some embodiments, the dose modification is a dose interruption. In some embodiments, the dose modification is a permanent discontinuation in dosing. In some embodiments, the dose modification is a dose reduction. In some embodiments, the dose of the compound represented by Formula (I) administered to the patient is reduced from 30 mg twice a week, e.g., one capsule comprising 30 mg of the anhydrous form of the compound represented by Formula (I) or, e.g., one capsule comprising 32.5 mg of the crystalline dihydrate form of the compound represented by Formula (I), to 20 mg twice a week, e.g., one capsule comprising 20 mg of the anhydrous form of the compound represented by Formula (I) or, e.g., one capsule comprising 21.7 mg of the crystalline dihydrate form of the compound represented by Formula (I). In some embodiments, the dose of the compound represented by Formula (I) administered to the patient is reduced from 30 mg twice a week, e.g., one capsule comprising 30 mg of the anhydrous form of the compound represented by Formula (I)) or, e.g., one capsule comprising 32.5 mg of the crystalline dihydrate form of the compound represented by Formula (I), to 14 mg twice a week, e.g., one capsule comprising 14 mg of the anhydrous form of the compound represented by Formula (I) or, e.g., one capsule comprising 15.2 mg of the crystalline dihydrate form of the compound represented by Formula (I). In some embodiments, the dose of the compound represented by Formula (I) administered to the patient is reduced from 20 mg twice a week, e.g., one capsule comprising 20 mg of the anhydrous form of the compound represented by Formula (I)) or, e.g., one capsule comprising 21.7 mg of the crystalline dihydrate form of the compound represented by Formula (I), to 14 mg twice a week, e.g., one capsule comprising 14 mg of the anhydrous form of the compound represented by Formula (I) or, e.g., one capsule comprising 15.2 mg of the crystalline dihydrate form of the compound represented by Formula (I). In some embodiments, the adverse reaction is selected from the group consisting of a hepatobiliary elevation and a treatment-related skin or subcutaneous disorder. In some embodiments, the treatment-related skin or subcutaneous disorder is selected from the group consisting of maculopapular rash, pruritus, urticaria, eczema, and dry skin.

A patient can also be administered an additional treatment prior to, or during administration of the compound represented by Formula (I) in accordance with the methods described herein to prevent or ameliorate an adverse event. In some embodiments, the patient is administered a topical composition (e.g., an emollient) before and/or during the compound represented by Formula (I) administration to prevent or ameliorate the onset of an adverse dermatologic reaction.

### EXAMPLES

### Example 1. A Phase 3, Randomized, Placebo-Controlled, Double-Blind Study of Vimseltinib in Patients with TGCT

This study is a two-part, randomized, double-blind, placebo-controlled study to assess the efficacy and safety of vimseltinib in patients with TGCT not amenable to surgery with no prior anti-CSF1/CSF1R therapy (prior therapy with imatinib or nilotinib allowed). In Part 1, patients (n=123) were randomized two-to-one to receive either 30 mg twice weekly of vimseltinib (n=83) or placebo (n=40) for 24 weeks. Vimseltinib or matching placebo was administered orally each week on day 1 and day 5 (±1 day) at approximately the same time of day on an empty stomach, with ≥24 hours between doses. An MRI of the affected joint was performed at screening, cycle 4 day 1 (week 13; ±7 days), and at the end of Part 1. Part 2 is an ongoing open-label study, in which patients from both the vimseltinib and placebo arms receive treatment with vimseltinib.

The primary endpoint of the study is the objective response rate (ORR) at Week 25 as measured by Response Evaluation Criteria in Solid Tumors (RECIST) version 1.1 by blinded independent radiologic review (IRR). Key secondary endpoints included the objective response rate at Week 25 by IRR per tumor volume score (TVS), active range of motion (ROM) at Week 25, PROMIS physical function (PROMIS-PF) score, worst stiffness numeric rating scale (NRS) score at Week 25, EQ-visual analogue scale (EQ-VAS) score at Week 25, and greater than or equal to 30% reduction in Brief Pain Inventory (BPI) in worst pain and less than 30% increase in narcotic use (BPI-30) at Week 25. Other secondary endpoints included duration of response (DOR) and safety.

Eligibility Criteria: Eligible patients were aged ≥18 years with a histologically confirmed diagnosis of TGCT for which surgical resection would potentially cause worsening functional limitation or severe morbidity. Patients had at least 1 measurable lesion ≥2 cm using Response Evaluation Criteria in Solid Tumors version 1.1 (RECIST v1.1), as assessed from magnetic resonance imaging (MRI) by a central radiologist.

Patients were excluded for receiving previous systemic therapy targeting CSF1 or CSF1R including vimseltinib; previous therapy with imatinib and/or nilotinib was allowed. Patients who received any therapy for TGCT (including investigational therapy) during screening, had major surgery 14 days prior to the first dose of study drug, or participated in a non-TGCT investigational drug study within 30 days of screening were also excluded.

The demographics and baseline disease characteristics of the ITT population is provided in Table 1 below.

TABLE 1

| | Vimseltinib | Placebo | Total |
|---|---|---|---|
| | (N = 83) | (N = 40) | (N = 123) |
| Age Median | 45 (20, 78) | 43 (21, 72) | 44 (20, 78) |
| (min, max) | | | |
| Gender: Male (%) | 37 (45) | 13 (33) | 50 (41) |
| Race: White (%) | 59 (71) | 21 (53) | 80 (65) |
| Region: US (%) | 9 (11) | 4 (10) | 13 (11) |
| non-US (%) | 74 (89) | 36 (90) | 110 (89) |
| Tumor Location | 73 (88) | 36 (90) | 109 (89) |
| per IRT: Lower Limb | | | |
| (Hip/Knee/Ankle) (%) | | | |
| All Other | 10 (12) | 4 (10) | 14 (11) |
| Primary Affected Joint | | | |
| Knee | 56 (67) | 27 (68) | 83 (67) |
| Ankle | 9 (11) | 6 (15) | 15 (12) |
| Hip | 11 (13) | 1 (3) | 12 (10) |
| Other | 7 (8) | 6 (15) | 13 (11) |
| Subtype: | 57 (69) | 28 (70) | 85 (69) |
| Diffuse TGCT | | | |
| Any Prior Surgery | 64 (77) | 27 (68) | 91 (74) |
| Any prior | 19 (23) | 9 (23) | 28 (23) |
| TGCT Systemic | | | |
| Therapy | | | |
| Imatinib | 16 (19) | 7 (18) | 23 (19) |
| Nilotinib | 2 (2) | 4 (10) | 6 (5) |
| Other | 1 (1) | 0 | 1 (1) |

### Endpoint Analyses

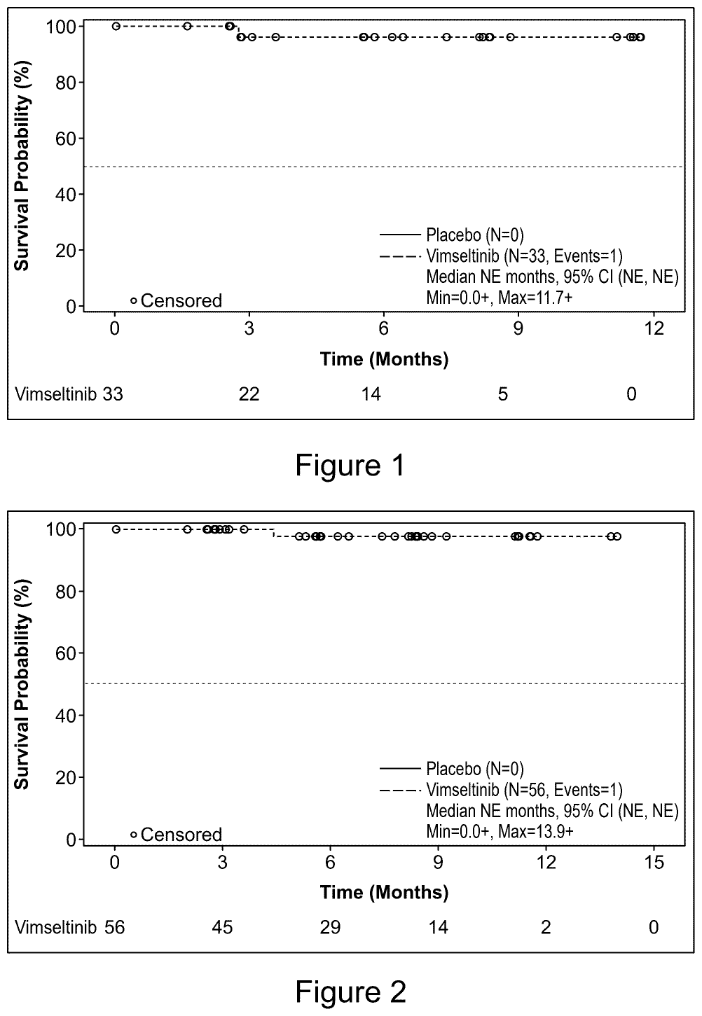

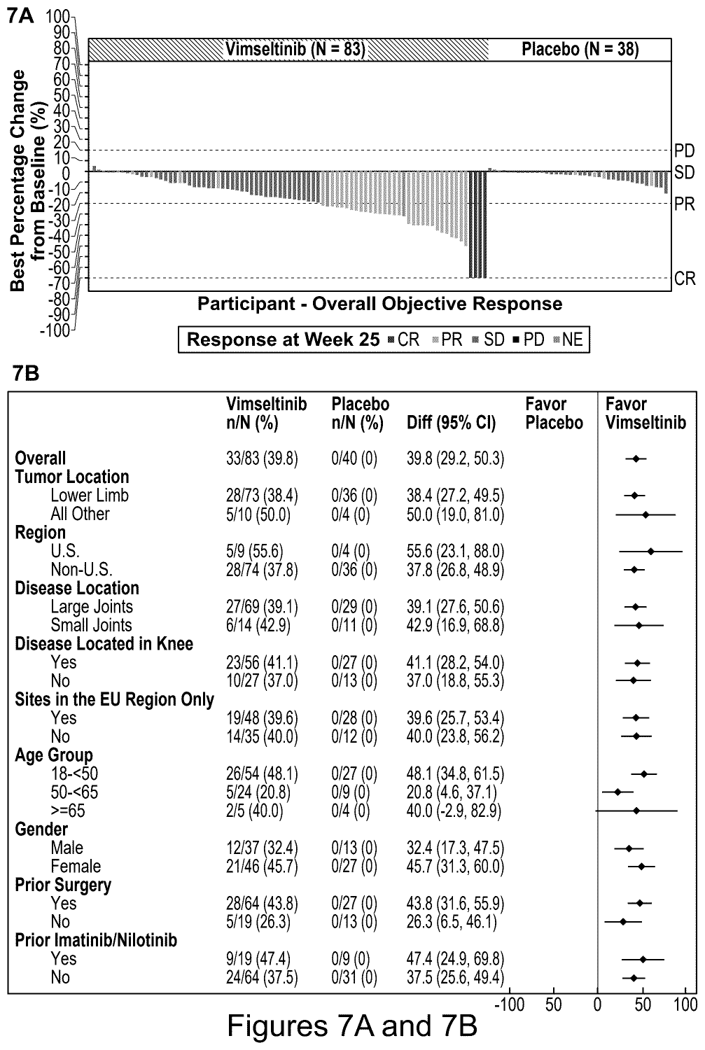

The study met its primary endpoint in the intent-to-treat (ITT) population demonstrating statistically significant and clinically meaningful improvement in the primary endpoint of ORR at Week 25 based on IRR per RECIST 1.1. In the ITT population, the ORR at Week 25 was 40% (95% CI: 29%, 51%) for the vimseltinib arm and 0% (95% CI: 0%, 9%) for the placebo arm resulting in a response difference (vimseltinib vs. placebo) of 40% (95% CI: 29%, 51%) (p<0.0001). Further, the median duration of response was not reached, where the range was (0.03+, 11.7+) months for vimseltinib (FIG. 1). Nearly all patients receiving vimseltinib experienced clinical benefit (CR+PR+SD) as shown in the best percentage change in target lesions as assessed by IRR in FIG. 7A. FIG. 7B shows that, when evaluating ORR by demographic characteristics, all groups favored treatment with vimseltinib. Analysis of the primary endpoint is provided in Table 2, showing that ORR at Week 25 by IRR per RECIST v1.1 was statistically significantly higher for vimseltinib than placebo (40% vs 0%, difference 40% [195% CI: 29%, 51%], 2-sided p<0.0001).

TABLE 2

| | Vimseltinib | Placebo |
|---|---|---|
| ITT Population | (N = 83) | (N = 40) |
| ORR at Week 25 by IRR | 33 (40%) | 0 (0%) |
| per RECIST 1.1 | | |
| n (%) (95% CI) | (29%, 51%) | (0%, 9%) |
| Complete Response (CR) | 4 (5%) | 0 (0%) |
| Partial Response (PR) | 29 (35%) | 0 (0%) |
| Stable disease (SD) | 42 (51%) | 33 (83%) |

| | |
|---|---|
| Difference in ORR % | 40% |
| (95% CI) | (29%, 51%) |
| CMH test p-value* | <0.0001 |

### Tumor Volume Score

Tumor volume score (TVS) is a semiquantitative magnetic resonance imaging scoring system that describes tumor mass. It is based on 10% increments of the estimated volume of the maximally distended involved synovial cavity or tendon sheath. Scores ranged from 0 to 10, and a tumor that was equal in volume to that of a maximally distended synovial cavity or tendon sheath was scored a 10; a score of 0 indicated no evidence of a tumor.

Complete response denoted disappearance of all tumor(s). Partial response was at least a 50% reduction from baseline in TVS. Progressive disease was at least a 30% increase in TVS from baseline. Stable disease was neither a sufficient decrease to qualify for partial response nor sufficient increase to qualify for progressive disease, taking the nadir as a reference. Response was considered not evaluable if the TVS could not be assessed for reasons such as inadequate or missing imaging.

Vimseltinib demonstrated a statistically significant and clinically meaningful improvement in ORR at Week 25 by IRR per TVS. In the ITT population, ORR at Week 25 by IRR per TVS was statistically significantly higher for vimseltinib than placebo (67% vs 0%, difference 67% [95% CI: 56%, 77%], 2-sided p<0.0001). The median duration of response was not reached, where the range was (0.03+, 13.9+) months for vimseltinib (FIG. 2). Analysis of this endpoint is provided in Table 3.

TABLE 3

| | Vimseltinib | Placebo |
|---|---|---|
| ITT Population | (N = 83) | (N = 40) |
| ORR at Week 25 by | 56 (67%) | 0 (0%) |
| IRR per TVS | | |
| n (%) (95% CI) | (56%, 77%) | (0%, 9%) |
| CR | 4 (5%) | 0 (0%) |
| PR | 52 (63%) | 0 (0%) |
| SD | 19 (23%) | 34 (85%) |

| | |
|---|---|
| Difference in ORR | 67% |
| % (95% CI) | (56%, 77%) |
| CMH test p-value* | <0.0001 |

### Active Range of Motion Calculation

Active range of motion (ROM; in degrees) was assessed using a goniometer and was normalized using a reference standard provided by the American Medical Association to compute the relative active ROM. In joints where more than one motion was measured, the motion with the smallest relative ROM value (worst) was used for evaluating subsequent changes in relative active ROM.

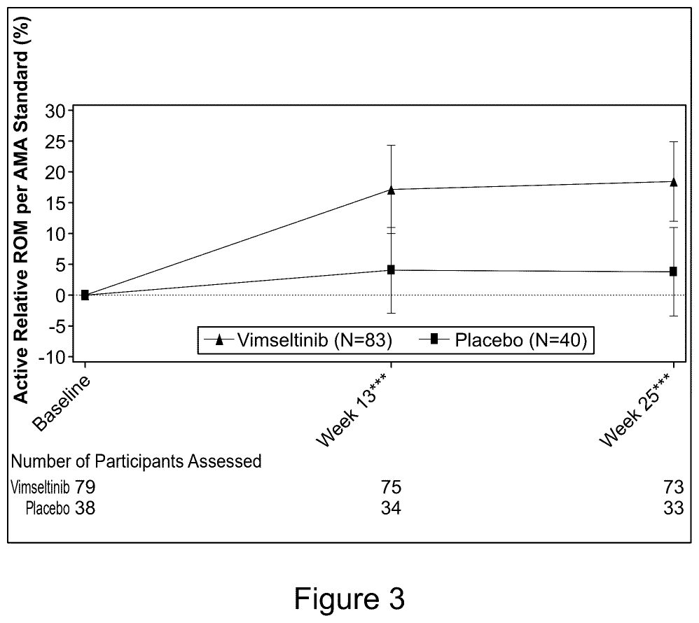

Vimseltinib demonstrated a statistically significant and clinically meaningful improvement in mean change from baseline (CFB) in active ROM at Week 25 (18.4% vs 3.8%, difference 14.6% [95% C: 4.0%, 25.3%], 2-sided p=0.0077). FIG. 3 displays the CFB in active ROM. Analysis of this endpoint is provided in Table 4. Change from baseline is modeled with fixed effects of treatment group, timepoint, treatment group by timepoint interaction, region (US vs. non-US), joint type (knee, ankle, or other), and baseline ROM.

TABLE 4

| | Vimseltinib | Placebo |
|---|---|---|
| ITT Population | (N = 83) | (N = 40) |
| Baseline Mean (SD) | 63.0% (29.37) | 62.9% (32.22) |
| CFB in Active ROM | | |
| at Week 25 | | |
| LS Mean (95% CI) | 18.4% (5.6, 31.2) | 3.8% (−10.5, 18.0) |
| Difference in LS | 14.6% (4.0, 25.3) | |
| Mean (95% CI) | | |
| p-value | 0.0077 | |

The response rate in active ROM at Week 25 was 48% for vimseltinib (N=79) and 20% for placebo (N=38) (difference 28% [95% CI: 12%, 45%], p=0.0025).

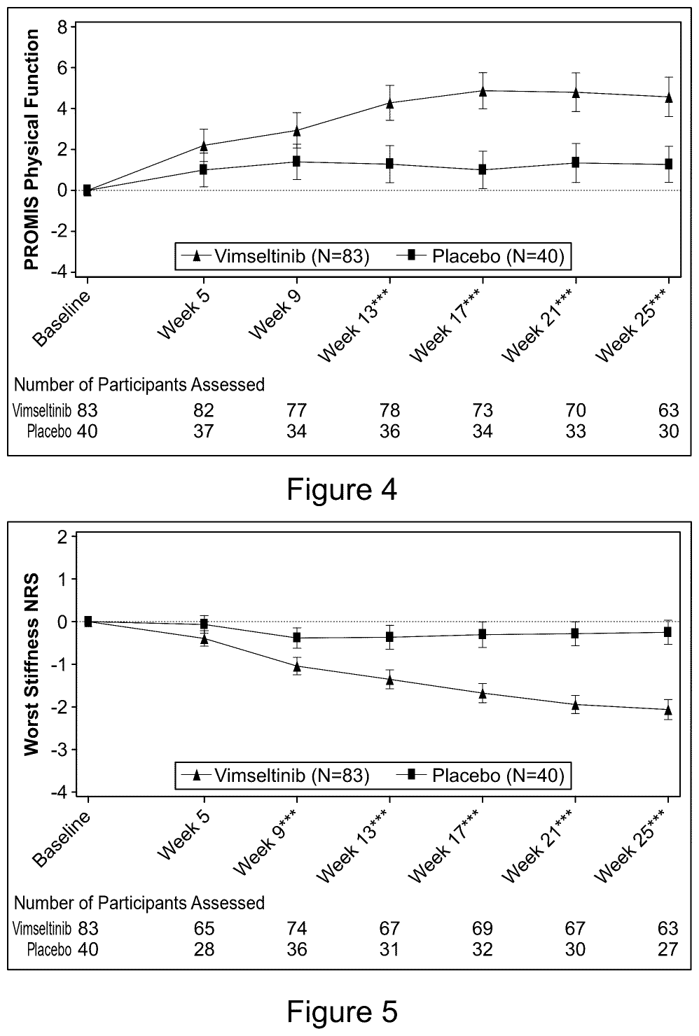

Vimseltinib demonstrated a statistically significant and clinically meaningful improvement in mean CFB in PROMIS-PF at Week 25 (4.6 vs 1.3, difference 3.3 [95% CI: 1.4, 5.2], 2-sided p=0.0007). FIG. 4 displays the CFB in PROMIS-PF. Analysis of this endpoint is provided in Table 5. Change from baseline is modeled with fixed effects of treatment group, timepoint, treatment group by timepoint interaction, region (US vs. non-US), tumor location (lower limb vs. all other), and baseline PROMIS-PF.

TABLE 5

| | Vimseltinib | Placebo |
|---|---|---|
| ITT Population | (N = 83) | (N = 40) |
| Baseline Mean (SD) | 39.0 (6.14) | 38.5 (5.98) |
| CFB in PROMIS-PF at Week 25 | | |
| LS Mean (95% CI) | 4.6 (2.7, 6.5) | 1.3 (−0.5, 3.0) |
| Difference in LS Mean (95% CI) | 3.3 (1.4, 5.2) | |
| p-value | 0.0007 | |

The response rate in PROMIS-PF at Week 25 was 43% for vimseltinib (N=79) and 25% for placebo (N=38) (difference 18% [95% CI: 1%, 36%], p=0.046).

Vimseltinib demonstrated a statistically significant and clinically meaningful improvement in mean CFB in Worst Stiffness NRS at Week 25 (−2.1 vs −0.3, difference −1.8 [95% CI: −2.5, −1.1], 2-sided p<0.0001). FIG. 5 displays the CFB in Worst Stiffness NRS. Analysis of this endpoint is provided in Table 6. Change from baseline is modeled with fixed effects of treatment group, timepoint, treatment group by timepoint interaction, region (US vs. non-US), tumor location (lower limb vs. all other), and baseline Worst Stiffness NRS.

TABLE 6

| | Vimseltinib | Placebo |
|---|---|---|
| ITT | (N = 83) | (N = 40) |
| Baseline Mean (SD) | 5.1 (2.00) | 5.2 (1.81) |
| CFB in Worst Stiffness | | |
| NRS at Week 25 | | |
| LS Mean (95% CI) | −2.1 | −0.3 |
| | (−2.5, −1.6) | (−0.8, 0.3) |
| Difference in LS | −1.8 | |
| Mean (95% CI) | (−2.5, −1.1) | |
| p-value | <0.0001 | |

The response rate in Worst Stiffness NRS at Week 25 was 39% for vimseltinib (N(79) and 15% for placebo (NN38) (difference 24% [95% C: 8%, 39%], p=0.0080).

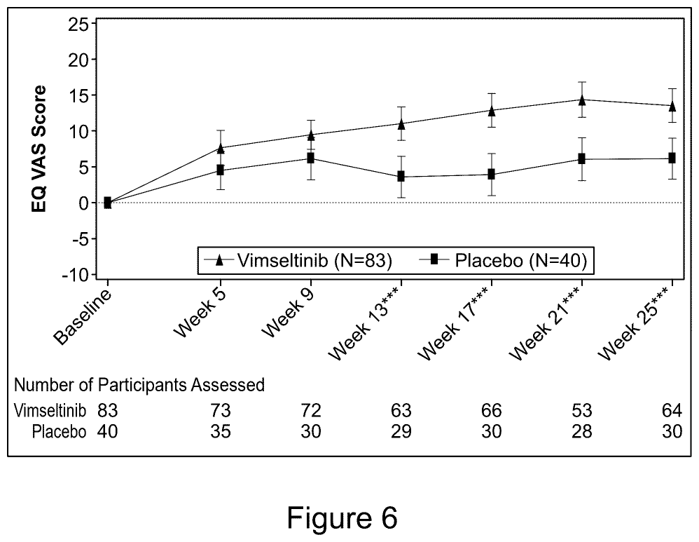

Vimseltinib demonstrated a statistically significant and clinically meaningful improvement in mean CFB in EQ-VAS at Week 25 (13.5 vs 6.1, difference 7.4 [95% Cl: 1.4, 13.4], 2-sided p=0.0155). FIG. 6 displays the CFB in EQ-VAS score. Analysis of this endpoint is provided in Table 7. Change from baseline is modeled with fixed effects of treatment group, timepoint, treatment group by timepoint interaction, region (US vs. non-US), tumor location (lower limb vs. all other), and baseline EQ-VAS.

TABLE 7

| | Vimseltinib | Placebo |
|---|---|---|
| ITT Population | (N = 83) | (N = 40) |
| Baseline Mean (SD) | 61.4 (19.53) | 60.2 (20.63) |
| CFB in EQ-VAS | | |
| at Week 25 | | |
| LS Mean (95% CI) | 13.5 | 6.1 |
| | (8.9, 18.2) | (0.5, 11.8) |
| Difference in LS | 7.4 | |
| Mean (95% CI) | (1.4, 13.4) | |
| p-value | 0.0155 | |

The response rate in EQ-VAS at Week 25 was 37% for vimseltinib (N=79) and 25% for placebo (N=38) (difference 12% [95% CI: −5%, 29%], p=0.16).

Vimseltinib demonstrated a statistically significant and clinically meaningful improvement in BPI-30 response rate in Worst Pain at Week 25 (48% vs 23%, difference 26% [95% CI: 4%, 42%], 2-sided p=0.0056). Analysis of this endpoint is provided in Table 8.

TABLE 8

| | Vimseltinib | Placebo |
|---|---|---|
| ITT | (N = 83) | (N = 40) |
| BPI-30 Response in Worst Pain | 40 (48) | 9 (23) |
| n (%) (95% CI) | (37, 59) | (11, 38) |

| | |
|---|---|
| Difference in BPI-30 Response | 26 (4, 42) |

in Worst Pain % (95% CI)

| | |
|---|---|
| CMH test p-value* | 0.0056 |

*stratified by stratification factors based on IRT. BPI: Brief Pain Inventory; BPI-30 Response in Worst Pain: ≥30% reduction in BPI worst pain NRS and <30% increase in narcotic use; CMH: Cochran-Mantel-Haenszel.

The response rate in BPI Worst Pain at Week 25 was 48% for vimseltinib (N=79) and 23% for placebo (N=38) (difference 26% [95% CI: 4%, 42%], p=0.0056).

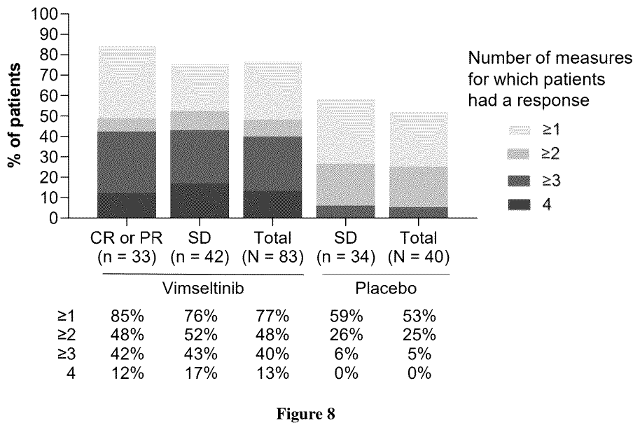

FIG. 8 shows the percent of patients experiencing functional and symptomatic improvement across active ROM, PROMIS-PF, worst stiffness NRS, and BPI worst pain response by treatment and REICST v1.1 tumor response at week 25. Response for PROMIS-PF, worst stiffness NRS, and EQ-VAS was a change from baseline ≥3, ≤−2, and ≥7, respectively. BPI worst pain response was defined as a ≥30% reduction in worst pain without a ≥30% increase in narcotic analgesic use. (CR, complete response; BPI, Brief Pain Inventory; NRS, numeric rating scale; PR, partial response; PRO, patient-reported outcome; PROMIS-PF; PRO Measurement Information System physical function; RECIST v1.1, Response Evaluation Criteria in Solid Tumors version 1.1; ROM; range of motion; SD, stable disease; TGCT, tenosynovial giant cell tumor). Symptomatic improvements were observed in patients receiving vimseltinib who had an objective tumor response or stable disease (SD) per RECIST v1.1. More patients experienced improvement in multiple measures with vimseltinib versus placebo.

### Safety

Vimseltinib was well-tolerated and the safety profile in the study was consistent with previously disclosed data. There was no evidence of cholestatic hepatotoxicity in patients treated with vimseltinib. Patients with treatment-emergent adverse events (TEAEs) leading to treatment discontinuation was 5% in the vimseltinib arm. The table below lists TEAEs greater than 15% in either arm during Part 1 of the study. Table 9 summarizes safety data from the study.

TABLE 9

| | Vimseltinb | | | Placebo | | |
|---|---|---|---|---|---|---|
| | (n = 83) | | | (n = 39)¹ | | |
| Preferred | All | Grade | | All | | Grade |
| Term n (%) | Grades | 3-4 | | Grades | | 3-4 |
| Periorbital edema\hat{ } | 37 (45%) | 3 | (4%) | 5 | (13%) | 0 |
| Fatigue | 27 (33%) | 0 | | 6 | (15%) | 0 |
| Face edema\hat{ } | 26 (31%) | 1 | (1%) | 3 | (8%) | 0 |
| Pruritus\hat{ } | 24 (29%) | 2 | (2%) | 3 | (8%) | 0 |
| Headache\hat{ } | 23 (28%) | 1 | (1%) | 10 | (26%) | 0 |
| Asthenia\hat{ } | 22 (27%) | 1 | (1%) | 9 | (23%) | 1 (3%) |
| Nausea | 21 (25%) | 0 | | 8 | (21%) | 1 (3%) |
| CPK increased | 20 (24%) | 8 | (10%) | 0 | | 0 |
| AST increased | 19 (23%) | 0 | | 1 | (3%) | 0 |
| Arthralgia | 16 (19%) | 0 | | 6 | (15%) | 1 (3%) |
| Rash | 16 (19%) | 0 | | 2 | (5%) | 0 |
| Rash maculo-papular\hat{ } | 16 (19%) | 1 | (1%) | 0 | | 0 |
| Edema peripheral | 15 (18%) | 0 | | 3 | (8%) | 0 |
| Hypertension | 14 (17%) | 4 | (5%) | 4 | (10%) | 1 (3%) |

| | | | | | |
|---|---|---|---|---|---|
| Diarrhea | 10 (12%) | 0 | 8 | (21%) | 1 (3%) |

¹Safety population includes 122 patients. One patient was randomized to placebo but did not receive study drug.
\hat{ }Denotes adverse events (AEs) without Grade 4 criteria per CTCAE v5.0.

Further, no severe adverse event (SAE) occurred in more than one patient. There was one related SAE of subcutaneous abscess. Additionally, Grade 3/4 TEAEs that were observed in greater than 5% were creatine phosphokinase (CPK) increased (10%).

Updated results after 6 months demonstrated sustained tumor responses with vimseltinib and a safety profile consistent with previous reports.

### Example 2. Results from a Phase 1/2 Study of Vimseltinib in TGCT

This is a Phase 1/2 open-label, multicenter study evaluating the safety and efficacy of vimseltinib in patients with TGCT. In Phase 1, 32 patients enrolled in three cohorts across multiple doses. In Phase 2, 66 patients enrolled in two cohorts at the recommended Phase 2 dose of 30 mg twice weekly: Cohort A (n=46) in TGCT patients with no prior anti-CSF1/CSF1R therapy (prior therapy with imatinib or nilotinib allowed); and Cohort B (n=20) in TGCT patients with prior anti-CSF1/CSF1R therapy (prior therapy with imatinib or nilotinib alone not eligible).

With regards to antitumor activity and treatment duration, 93 patients were evaluable for efficacy by RECIST version 1.1 at the data cutoff date. Table 10 displays the response data based on IRR.

TABLE 10

| | | Phase 2 | Phase 2 |
|---|---|---|---|
| | Phase 1 | Cohort A | Cohort B |
| | (n = 32) | (n = 45) | (n = 16) |
| ORR per RECIST v1.1 by IRR | 72% | 64% (38% at | 44% |
| (%) | | Week 25) | |
| Median Duration of Response | NR (3.8+, | NR (0.03+, | NR (4.0+, |
| (months) (Range) | 45.2+) | 25.4+) | 21.0+) |
| Median Treatment Duration | 25.1 | 21.0 | 7.3 |
| (months) (Range) | (0.7, 46.9) | (0.2, 30.3) | (0.7, 27.4) |
| Patients Active at Cutoff | 47% | 48% | 74% |
| Date (%) | | | |

NR: Not Reached

In addition, updated data from Cohorts A and B of the Phase 2 study demonstrate that patients experienced clinically meaningful symptomatic benefit at Week 25 across multiple secondary efficacy measures including ORR by TVS (Cohort A), active range of motion, physical function, stiffness, and pain.

With regards, to safety and tolerability, treatment with vimseltinib was well-tolerated in patients with TGCT and consistent with previously disclosed data, there was no evidence of cholestatic hepatotoxicity, and treatment discontinuation due to TEAEs occurred in 9% of patients.

Longer follow-up demonstrated that vimseltinib continued to be well tolerated with a manageable safety profile in patients with TGCT not amendable to surgery who received or did not receive prior anti-CSF1/CSF1R therapy. Table 11 displays the response data for the second cutoff date for Cohorts A and B based on IRR.

TABLE 11

| | Phase 2 | Phase 2 |
|---|---|---|
| | Cohort A | Cohort B |
| | (n = 45) | (n = 19) |
| ORR per RECIST v1.1 | 64% (38% | 37% (26% |
| by IRR (%) | at Week 25) | at Week 25) |
| ORR per TVS by IRR (%) | 62% (51% | 42% (26% |
| | at Week 25) | at Week 25) |
| Median Duration of Response | NR (0.7+, | NR (4.0+, |
| (months) (Range) | 33.4+) | 29.5+) |
| Median Treatment Duration | 22.2 | 11.1 |
| (months) (Range) | (0.2, 36.6) | (0.7, 36.1) |
| Patients Active at Cutoff | 41% | 65% |
| Date (%) | | |

NR: Not Reached

As of the second data cutoff, no patients progressed as assessed by IRR. Further, most patients in Cohort B experienced an increase in active ROM as of the second data cutoff, as shown in Table 12 below. In Table 12, the analysis only includes patients with active ROM assessments at baseline and Week 25.

TABLE 12
Cohort B (n = 13) - Active ROM
of the affected joint, mean (SD), %

| | |
|---|---|
| Baseline | 51.1 (35.9) |
| Week 25 | 65.2 (37.5) |
| Change from baseline to Week 25, | 14.1 (27.4) |
| mean (SD), percentage points | |

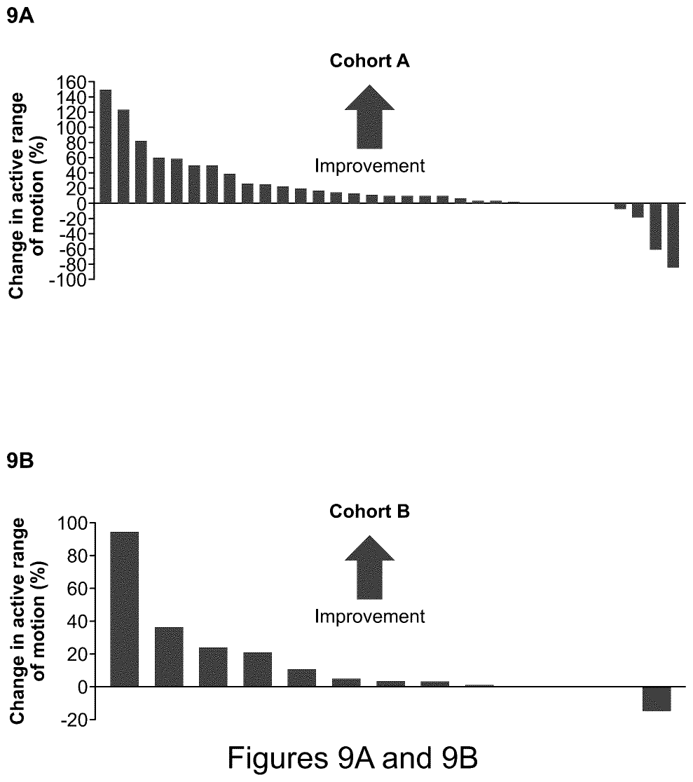

Further, the majority of patients in cohort A experienced an increase in active ROM, and the mean change from baseline (standard deviation [SD]) at week 25 was 19.7 (43.5) percentage points. FIG. 9A depicts that the majority of patients in cohort A experienced an increase in active ROM, and the mean change from baseline (standard deviation [SD]) at week 25 was 19.7 (43.5) percentage points.

### Patient Reported Outcomes (Cohort B)

Patient-reported outcomes for Cohort B patients at Week 25 of receiving vimseltinib were obtained. At Week 25, 55% (11/20) of patients experienced BPI response (defined as ≥30% reduction in pain without a ≥30% increase in narcotic analgesic use) for both worst pain and average pain. Two patients (40%) with objective responses by RECIST v 1.1 at Week 25 were also BPI responders. Most patients (78%) with stable disease at Week 25 were also BPI responders, which includes patients with both BPI and efficacy data available at week 25 (n =14), where percentages represent proportion of patients with partial response or stable disease with ≥30% pain reduction.

At Week 25, 63% and 58% of patients had clinically meaningful improvements in PROMIS-PF and numeric rating scale (NRS) stiffness, respectively.

Further, Table 13 shows a summary of clinical outcome assessments at Week 25 at the second cutoff date noted above:

TABLE 13

| | Cohort A | Cohort B |
|---|---|---|
| | n = 46 | n = 20 |
| Active ROM^{a} | | |

| | | |
|---|---|---|
| Patients, n | 33 | 13 |

| | | | | |
|---|---|---|---|---|
| Baseline, mean (SD), % | 58.1 | (32.4) | 51.1 | (35.9) |
| Week 25, mean (SD), % | 77.8 | (39.0) | 65.2 | (37.5) |
| Change from baseline, | 19.7 | (43.5) | 14.1 | (27.4) |
| mean (SD), percentage | | | | |
| points | | | | |
| Response rate^{b}, No. (%) | 19 | (42) | 5 | (26) |
| PROMIS-PF^{c} | | | | |

| | | |
|---|---|---|
| Patients, n | 35 | 15 |

| | | | | |
|---|---|---|---|---|
| Baseline, mean (SD) | 40.0 | (8.2) | 43.3 | (8.0) |
| Week 25, mean (SD) | 43.9 | (9.1) | 49.5 | (7.8) |
| Change from baseline, | 3.9 | (7.8) | 6.2 | (8.0) |
| mean (SD) | | | | |
| Response rate^{d}, No. (%) | 17 | (49) | 12 | (80) |
| Worst stiffness NRS^{e} | | | | |

| | | |
|---|---|---|
| Patients, n | 32 | 14 |

| | | | | |
|---|---|---|---|---|
| Baseline, mean (SD) | 4.9 | (2.1) | 4.7 | (2.4) |
| Week 25, mean (SD) | 3.1 | (2.2) | 2.6 | (2.2) |
| Change from baseline, | −1.8 | (2.7) | −2.2 | (3.0) |
| mean (SD) | | | | |
| Response rate^{f}, n (%) | 21 | (66) | 10 | (71) |
| BPI worst pain | | | | |

| | | |
|---|---|---|
| Patients, n | 46 | 20 |

| | | | | |
|---|---|---|---|---|
| Response rate^{g}, No. (%) | 23 | (50) | 11 | (55) |

^{a}Includes patients with active ROM values and baseline and week 25.
^{b}Response was defined as at least a 10% increase at week 25.
^{c}Includes patients with PROMIS-PF values at baseline and week 25.
^{d}Response was defined as at least a 3-point increase at week 25.
^{e}Includes patients with worst stiffness NRS values at baseline and week 25.
^{f}Response was defined as at least a 2-point decrease at week 25.
^{g}Response was defined as a 30% or greater decrease in mean BPI worst pain without a 30% or greater increase in narcotic analgesic use.
Abbreviations: BPI, Brief Pain Inventory; NRS, Numeric Rating Scale; PROMIS-PF, Patient-Reported Outcome Measurement Information System Physical Function; ROM, range of motion; SD, standard deviation.

Table 14 below shows results for BPI Worst Pain by objective response per RECIST v1.1 at Week 25.

TABLE 14

| | Cohort A | | Cohort B | |
|---|---|---|---|---|
| | RECIST v1.1 Response | | RECIST v.1.1 Response | |
| | at Week 25 by IRR^{a} | | at Week 25 by IRR^{b} | |
| | Partial | Stable | Partial | Stable |
| | Response | Disease | Response | Disease |
| | (n = 17) | (n = 22) | (n = 5) | (n = 9) |
| Worst pain | 10 (59) | 12 (55) | 2 (40) | 7 (78) |
| responder^{c}, | | | | |
| No. (%) | | | | |
| Average pain | 12 (71) | 12 (55) | 2 (40) | 7 (78) |
| responder^{c}, | | | | |
| No. (%) | | | | |

^{a}Includes patients with both BPI and efficacy data available at week 25 (n = 39).
^{b}Includes patients with both BPI and efficacy data available at week 25 (n = 14).
^{c}Response defined as a ≥30% decrease in pain without a ≥30% increase in narcotic analgesic use.
Abbreviations: BPI, Brief Pain Inventory; IRR, independent radiological review; RECIST v1.1, Response Evaluation Criteria in Solid Tumors version 1.1.

Table 15 below shows results for Worst Stiffness NRS Response by Objective Response per RECIST v1.1 at Week 25.

TABLE 15

| | Cohort A | | Cohort B | |
|---|---|---|---|---|
| | RECIST v1.1 Response | | RECIST v.1.1 Response | |
| | at Week 25 by IRR^{a} | | at Week 25 by IRR^{b} | |
| | Partial | Stable | Partial | Stable |
| | Response | Disease | Response | Disease |
| | (n = 17) | (n = 22) | (n = 5) | (n = 9) |
| Worst pain | 10 (59) | 12 (55) | 2 (40) | 7 (78) |
| responder^{c}, | | | | |
| No. (%) | | | | |
| Average pain | 12 (71) | 12 (55) | 2 (40) | 7 (78) |
| responder^{c}, | | | | |
| No. (%) | | | | |

^{a}Includes patients with both BPI and efficacy data available at week 25 (n = 39).
^{b}Includes patients with both BPI and efficacy data available at week 25 (n = 14).
^{c}Response defined as a ≥30% decrease in pain without a ≥30% increase in narcotic analgesic use.
Abbreviations: BPI, Brief Pain Inventory; IRR, independent radiological review; RECIST v1.1, Response Evaluation Criteria in Solid Tumors version 1.1.

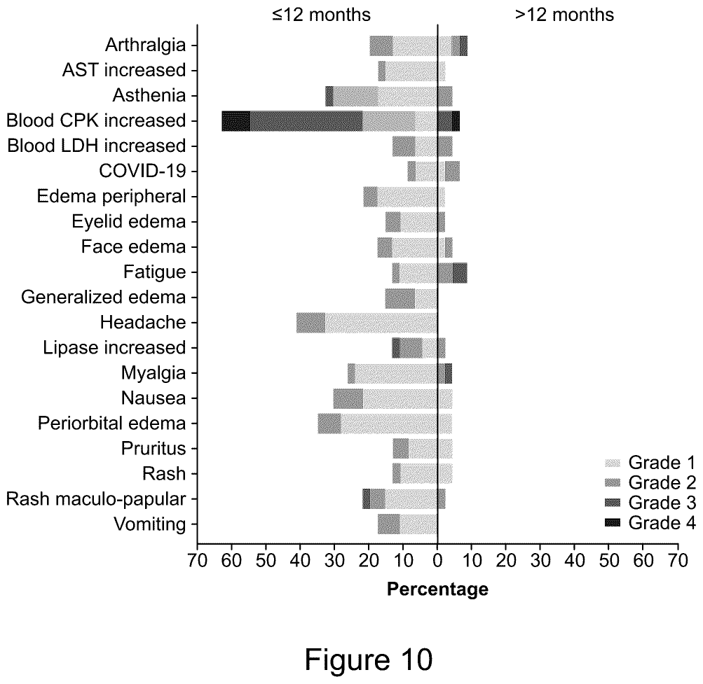

The majority of TEAEs were grade 1 or 2 (Table 2). Grade 3/4 TEAEs in >5% of patients in cohort A were increased blood creatine phosphokinase (CPK; 48%) and hypertension (9%); in Cohort B, these were increased blood CPK (35%), hypertension (15%), and eczema (10%). There was no evidence of cholestatic hepatotoxicity or drug-induced liver injury. Median (range) treatment duration was 22.2 (0.2-36.6) months for cohort A and 11.1 (0.7-36.1) months for Cohort B. The safety profile was consistent with continued treatment; the onset of the majority of the most severe events occurred within the first 12 months of treatment, as shown by FIG. 10.

### Example 3. Dose Interruption and Modification of Vimseltinib

This is dose interruption and modification protocol in a Phase 3, randomized, placebo-controlled, double-blind study of vimseltinib to assess the efficacy and safety in Patients with tenosynovial giant cell tumor. Administration of vimseltinib may be interrupted and/or modified at the discretion of the Investigator at any time due to adverse events (AEs). Vimseltinib may also be interrupted for other reasons in consultation with the Sponsor Medical Monitor. Any vimseltinib interruption unrelated to an AE will be limited to 28 days. Upon resumption of vimseltinib following a dose interruption, the Investigator must continue with the participant's original visit schedule calculated from Cycle 1 Day 1. Clinic visits and assessments should continue during dose interruptions.

### Severity Assessment

The Investigator must determine and record the severity of all serious and nonserious AEs. The NCI-CTCAE, v5.0, must be used for grading the severity of AEs (NCI-CTCAE, 2020). When there is a change in severity of an existing adverse event including improvement or worsening of an event, a new AE should be reported.

The severity of an AE that does not appear in the CTCAE scale must be determined according to Table 16.

TABLE 16
Severity Grading Scale

| Classification | Definition |
|---|---|
| Grade 1 (Mild) | Asymptomatic or mild symptoms; clinical |
| | or diagnostic observations only; |
| | intervention not indicated. |
| Grade 2 | Minimal, local, or noninvasive intervention |
| (Moderate) | indicated; limiting age-appropriate instrumental |
| | Activities of Daily Living. |
| Grade 3 | Severe or medically significant but not immediately |
| (Severe) | life-threatening; hospitalization or prolongation of |
| | hospitalization indicated; disabling; limiting self-care |
| | Activities of Daily Living. |
| Grade 4 (Life | Life-threatening consequences; urgent |
| threatening) | intervention indicated. |
| Grade 5 | Death related to AE. |
| (Death) | |

Abbreviation: AE = adverse event

### Dose Interruption and Modification

Vimseltinib must be interrupted for treatment-related Grade 3 AEs of any duration with the following exceptions: Asymptomatic/nonclinically significant Grade ≥3 elevation of CPK not accompanied by myalgias, worsening renal function, signs of rhabdomyolysis or cardiac muscle damage (e.g., myoglobinuria, CPK isozyme profile, troponin), and not requiring additional evaluation/testing; treatment-related Grade 3 nausea, vomiting, and diarrhea resolved to Grade 1 or baseline within 2 days with optimal treatment; treatment-related transient Grade 3 fatigue (lasting less than 3 days); treatment-related transient (lasting less than 3 days) Grade >3 laboratory abnormalities that resolve with optimal supplementation or support

For the treatment-related Grade 3 AEs resulting in dose interruptions, vimseltinib may be resumed at the same dose, or dose reduced per Investigator discretion, when the toxicity has recovered to Grade 1 or baseline, unless otherwise specified in management tables.

For all other treatment-related AEs resulting in dose interruptions, vimseltinib may be resumed at the same dose, unless otherwise specified in management tables.

Vimseltinib will be permanently discontinued for participants with treatment-related Grade 4 AEs except transient laboratory abnormalities as described above and Grade 4 neutropenia lasting for 7 days or fewer. If, in the opinion of the Investigator and the Sponsor, it is in the participant's best interest to continue treatment with vimseltinib, then the dose of vimseltinib will be reduced by at least 1 dose level when treatment resumes, after recovery of the toxicity or toxicities in question to Grade 1 or to baseline values.

### Dose Reduction Steps

If dose reduction is required, dose reduction steps are as described in Table 17.

TABLE 17
Dose Reduction Steps for Vimseltinib and Vimseltinib Dihydrate

| | Equivalent | | |
|---|---|---|---|
| | Amount of | | |
| Dose of | Vimseltinib | | Reduced |
| Vimseltinib | Dihydrate | | Vimseltinib |
| at the Time | at the Time | Reduced | Dihydrate |
| of Dose | of Dose | Vimseltinib | Equivalent |
| Modification | Modification | Dose | Amount |
| 30 mg | 32.5 mg | 20 mg | 21.7 mg |
| 20 mg | 21.7 mg | 14 mg | 15.2 mg |

If a dose reduction of vimseltinib is required, re-escalation of dose may be permitted based on agreement between the Investigator and Sponsor.

A participant will be allowed to have 2 dose level reductions. If more than 2 dose level reductions are required, the vimseltinib will be discontinued.

### Dose Interruption and Management of Toxicities

### Guidelines for Hepatobiliary Lab Elevations

Recommended dose interruption and management of treatment-related hepatobiliary lab elevations are shown in Table 18.

TABLE 18
Dose Interruption and Management of Treatment-related
Hepatobiliary Laboratory Elevations

| Toxicity Grade CTCAE v5.0 | Dose Interruption and Management |
|---|---|
| Grade 2 ALT and/or AST increase | Continue treatment with vimseltinib |
| (>3-5 × ULN) and total | Check for changes to medications and symptoms |
| bilirubin ≤ ULN | Perform confirmation liver enzyme (AST, ALT and |
| | ALP) and bilirubin tests within 48-72 hours |
| | Repeat liver enzyme tests weekly for 8 weeks to |
| | ensure stability |
| Grade 2 ALT and/or AST increase | Hold treatment with vimseltinib |
| (>3-5 × ULN) and total bilirubin | Check for changes to medications and symptoms |
| increase up to 2 × ULN OR | Perform confirmation liver enzyme (AST, ALT, |
| Total bilirubin increase | and ALP) and bilirubin tests within 48-72 hours |
| up to 2 × ULN | Repeat liver enzyme tests weekly |
| | Vimseltinib may be resumed at 1 dose level |
| | reduction once Hy's law has been definitively |
| | ruled out, labs resolve to Grade 1 or baseline, after |
| | discussion with Sponsor |
| | If ALT/AST/bilirubin continue to increase, or the |
| | increased level persists more than 28 days: |
| | Discontinue vimseltinib |
| | Refer for gastroenterology/hepatology consultation |
| | Consider liver ultrasound |
| Grade 2 ALT and/or AST increase | Hold treatment with vimseltinib |
| (>3-5 × ULN) and total bilirubin | Check for changes to medications and symptoms |
| increase >2 × ULN or INR >1.5 and | Perform confirmation liver enzyme (AST, ALT and |
| ALP <2 × ULN OR | ALP), bilirubin, and INR tests within 48-72 hours |
| Total bilirubin increase >2 × ULN | Refer for gastroenterology/hepatology consultation |
| | Consider liver ultrasound |
| | Repeat liver enzyme, bilirubin, and INR tests |
| | weekly until resolution to Grade 1 or baseline |
| | Vimseltinib may be resumed at 1 dose level |
| | reduction once Hy's law has been definitively ruled |
| | out, labs resolve to Grade 1 or baseline, after |
| | discussion with Sponsor |
| | If ALT/AST/bilirubin/INR continue to increase, or |
| | the increased level persists more than 28 days: |
| | Discontinue vimseltinib |
| Grade 3 ALT and/or AST increase | Hold treatment with vimseltinib |
| (>5-8 × ULN) and total | Check for changes to medications and symptoms |
| bilirubin ≤ ULN and without | Perform confirmation liver enzyme (AST, ALT and |
| clinical symptoms | ALP) and bilirubin tests within 48-72 hours |
| | Repeat liver enzyme tests weekly until resolution to |
| | Grade 1 or baseline |
| | Vimseltinib may be resumed at 1 dose level |
| | reduction once labs resolve to Grade 1 or baseline, |
| | after discussion with Sponsor |
| | If ALT/AST/bilirubin continue to increase, or the |
| | increased level persists more than 28 days: |
| | Discontinue vimseltinib |
| | Refer for gastroenterology/hepatology consultation |
| | Perform liver ultrasound |
| Grade 3 ALT and/or AST increase | Discontinue treatment with vimseltinib |
| (>5-8 × ULN) and total bilirubin | Check for changes to medications and symptoms |
| increase > ULN or | Perform confirmation liver enzyme (AST, ALT, and |
| INR >1.5 or ALP >2 × ULN | ALP), bilirubin, and INR tests within 48-72 hours |
| | Consider gastroenterology/hepatology consultation |
| | Perform liver ultrasound |
| | Repeat liver enzyme, bilirubin, and INR tests at least |
| | twice weekly until resolution to Grade 1 or baseline |
| Grade ≥3 ALT and/or AST increase | Discontinue treatment with vimseltinib |
| (>8 × ULN) | Check for changes to medications and symptoms |
| | Repeat liver enzymes and bilirubin tests at least |
| | twice weekly until resolution to Grade ≤2 |
| | Refer for gastroenterology/hepatology consultation |
| | Perform liver ultrasound |

Abbreviations: ALP = alkaline phosphatase; ALT = alanine aminotransferase; AST = aspartate aminotransferase; CTCAE = Common Terminology Criteria for Adverse Events; INR = international normalized ratio; ULN = upper limit of normal.
Note:
Cases of Hy's law must be reported as a serious adverse event and vimseltinib must be discontinued immediately.

### Guidelines for Prolonged QTcF Interval

TABLE 19
Recommended Dose Interruption and Management
of Treatment-related Prolonged QTcF Interval

| Toxicity Grade CTCAE | |
|---|---|
| v5.0 (ECG QTc interval | |
| prolonged) | Dose Interruption and Management |
| Grade 2 (QTc 481-500 ms) | Continue vimseltinib |
| | Continue ECG monitoring |
| | per protocol |
| Grade 3 (QTc ≥501 | Hold treatment with vimseltinib |
| ms; >60 ms change from | Continue close ECG monitoring |
| baseline) | and consult cardiologist |
| | Upon resolution to Grade 1 or |
| | baseline in ≤14 days, may resume |
| | study treatment at same dose. |
| | If >14 days for resolution to |
| | Grade 1 or baseline, may resume |
| | study treatment at reduced dose. |
| Grade 4 | Discontinue vimseltinib |
| | Continue close ECG monitoring |
| | and consult cardiologist |

Abbreviations: CTCAE = Common Terminology Criteria for Adverse Events; ECG = electrocardiogram; QTc = QT interval corrected; QTcF = QT interval corrected by Fridericia's formula.

### Guidelines for Dermatologic Adverse Events

Recommended dose interruption and management of treatment-related skin and subcutaneous disorder and recommendations for management of dry skin are presented in Table 20.

General guidelines for treatment-related dermatologic adverse events: Symptoms and signs of hypersensitivity reaction should be monitored at each clinic visit; use “Rule of 9s” (refer to Appendix 2) to calculate body surface area (BSA) for CTCAE grading; participant should be advised to use fragrance-free detergents and soaps, wear sunscreen with sun protection factor ≥30, and moisturize the skin at least once daily, preferably twice daily; standardized photos for any Grade ≥2 dermatologic AEs should be submitted to the Sponsor; skin biopsy sample (ie, punch biopsy) may be collected locally if a participant experiences a treatment-related dermatologic AE. If collected, the sample will be shipped to a central laboratory and may be analyzed for histopathology and potentially for molecular markers. Specific instructions on collection and shipping of samples will be provided in a separate laboratory manual.

TABLE 20
Recommended Dose Interruption and Management of Treatment-related Adverse Events

| Toxicity | | | | |
|---|---|---|---|---|
| Grade | | | | |
| (CTCAE | Maculopapular | | | |
| v5.0) | Rash | Pruritus/Urticaria | Eczema | Dry Skin |
| Grade 1 | Continue treatment | Continue treatment | Continue treatment | Continue treatment |
| | with vimseltinib | with vimseltinib | with vimseltinib | with vimseltinib |
| | Treat with mid- | Treat with cooling | Treat with mid- | Apply moisturizing |
| | to high-potency | lotions containing | to high-potency | creams/lotions |
| | topical | menthol and/or | topical | without |
| | corticosteroid bid | camphor | corticosteroid bid | fragrances/irritants |
| | | Treat with mid- | | (e.g., Oncoderm |
| | | to high-potency | | Body Rx spray; |
| | | topical | | Eucerin original) |
| | | corticosteroid bid | | |
| Grade 2 | Continue treatment | Continue treatment | Continue treatment | Continue treatment |
| | with vimseltinib | with vimseltinib | with vimseltinib | with vimseltinib |
| | Treat with high- | Treat with high- | Treat with high- | Apply moisturizing |
| | potency topical | potency topical | potency topical | creams/lotions |
| | corticosteroid | corticosteroid bid | corticosteroid bid | without |
| | 1-2 times per day | Initiate anti-H1 | Treat with anti-H1 | fragrances/irritants |
| | Treat with | therapy or | therapy or GABA | (e.g., Oncoderm |
| | anti-H1 therapy | GABA agonists | agonists (if eczema | Body Rx spray; |
| | (if rash is | Standardized | associated with | Eucerin original) |
| | associated with | photographs^{a} | pruritis) | |
| | symptoms such | Dermatologic | Standardized | |
| | as pruritus and/or | consultation | photographs^{a} | |
| | urticaria) or GABA | recommended | Dermatologic | |
| | agonists | | consultation | |
| | Standardized | | recommended | |
| | photographs^{a} | | | |
| | Dermatologic | | | |
| | consultation | | | |
| | recommended | | | |
| Grade 3 | Hold treatment | Hold treatment | Hold treatment | Hold treatment |
| | with vimseltinib | with vimseltinib | with vimseltinib | with vimseltinib |
| | Check | Standardized | Standardized | Use mid-potency |
| | CBC/differential- | photographs^{a} | photographs^{a} | steroid (e.g., |
| | evaluate for | Refer for | Refer for | triamcinolone |
| | eosinophilia | dermatologic | dermatologic | acetonide 0.1%) |
| | Standardized | consultation and | consultation and | bid plus |
| | photographs^{a} | obtain skin biopsy | obtain skin biopsy | moisturizing |
| | Refer for | Consider anti-IgE | Treat with oral | creams/lotion |
| | dermatologic | antibody following | corticosteroids | without |
| | consultation and | dermatologic | following | fragrances/irritants |
| | obtain skin biopsy | evaluation | dermatologic | (e.g., Oncoderm |
| | Treat with oral | Once AE resolves | evaluation | Body Rx spray; |
| | corticosteroids | to Grade ≤1, | (prednisone 0.5 | Eucerin original) |
| | following | restart vimseltinib | mg/kg/day) for | Once AE resolves |
| | dermatologic | at 1 dose level | 10-14 days | to Grade ≤1, |
| | evaluation | reduction | Consider | restart vimseltinib |
| | (prednisone | | interleukin-4 | at 1 dose level |
| | 0.5 mg/kg/day) | | receptor alpha | reduction |
| | for 10-14 days | | antagonist | |
| | Once AE resolves | | following | |
| | to Grade ≤1, | | dermatologic | |
| | may resume | | evaluation | |
| | vimseltinib at | | Once AE resolves | |
| | 1 dose level | | to Grade ≤1, | |
| | reduction | | restart vimseltinib | |
| | | | at 1 dose level | |
| | | | reduction | |

Abbreviations: AE = adverse event; bid = twice daily; CBC = complete blood count; CTCAE = Common Terminology Criteria for Adverse Events; GABA = gamma- aminobutyric acid; H1 = histamine-1; IgE = immunoglobulin E.
^{a}Guidance for standardized photographs will be provided separately

1. A method of treating a patient suffering from Grade 3 or 4 periorbital edema, peripheral edema, or face edema, while being administered a crystalline dihydrate form in an amount sufficient to provide 30 mg twice weekly of a compound represented by Formula (I): 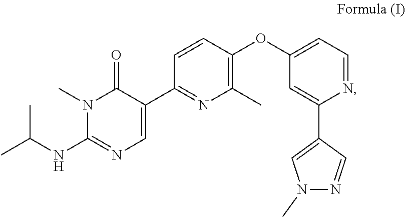 [SMILES: Cc1nc(-c2cnc(NC(C)C)n(C)c2=O)ccc1Oc1ccnc(-c2cnn(C)c2)c1]
for the treatment of tenosynovial giant cell tumor, and where surgical resection of the tumor would cause worsening functional limitation or severe morbidity, comprising administering a crystalline dihydrate form in an amount sufficient to provide twice weekly to the patient a dose of 20 mg or a dose of 14 mg of the compound.

2. The method of claim 1, wherein the patient is suffering from periorbital edema.

3. A method of treating a patient suffering from a treatment-emergent Grade 3 or 4 adverse reaction, while being administered a crystalline dihydrate form in an amount sufficient to provide 30 mg twice weekly of a compound represented by Formula (I): 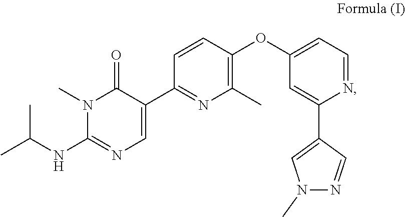 [SMILES: Cc1nc(-c2cnc(NC(C)C)n(C)c2=O)ccc1Oc1ccnc(-c2cnn(C)c2)c1]
for the treatment of tenosynovial giant cell tumor, and where surgical resection of the tumor would cause worsening functional limitation or severe morbidity, comprising administering twice weekly to the patient a crystalline dihydrate form in an amount sufficient to provide a dose of 20 mg or a dose of 14 mg of the compound.

4. A method of treating a patient for tenosynovial giant tumor, where surgical resection of the patient's tenosynovial giant tumor would cause worsening functional limitation or severe morbidity in the patient, comprising administering to the patient a crystalline dihydrate form in an amount sufficient to provide 30 mg twice weekly of a compound represented by Formula (I): 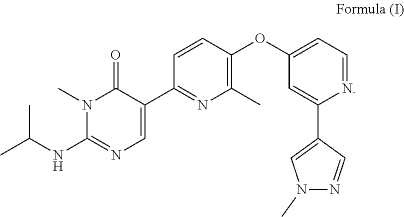 [SMILES: Cc1nc(-c2cnc(NC(C)C)n(C)c2=O)ccc1Oc1ccnc(-c2cnn(C)c2)c1]
wherein the patient suffers from a Grade 3 or 4 treatment-emergent periorbital edema during the administration, and the method further comprises then administering a crystalline dihydrate form in an amount sufficient to provide a first dose reduction of 20 mg of the compound twice weekly and if needed, administering a second dose reduction of 14 mg of the compound twice weekly to the patient.

5. The method of claim 3, wherein the Grade 3 or 4 treatment-emergent adverse event is periorbital edema.

6. The method of claim 3, wherein the Grade 3 or 4 treatment-emergent adverse event is peripheral edema.

7. The method of claim 3, wherein the Grade 3 or 4 treatment-emergent adverse event is face edema.
